(19) United States
(12) Patent Application Publication
(10) Pub. No.: US 2025/0087372 A1
(43) Pub. Date: Mar. 13, 2025

(54) SOURCE DATA REVIEW SYSTEM

(71) Applicant: IQVIA Inc., Parsippany, NJ (US)

(72) Inventors: Virupaxkumar Bonageri, Bangalore (IN); Rajneesh Patil, Durham, NC (US); Jonas Renström, Uppsala (SE); Jonathan Hill, San Diego, CA (US)

(21) Appl. No.: 18/883,806

(22) Filed: Sep. 12, 2024

Related U.S. Application Data
(60) Provisional application No. 63/582,387, filed on Sep. 13, 2023.

(51) Int. Cl. G16H 50/70; G06F 40/284; G06F 40/40; G16H 10/20

(52) U.S. Cl. CPC G16H 50/70; G06F 40/284; G06F 40/40; G16H 10/20

(57) ABSTRACT

Methods, systems, and apparatus, including computer programs encoded on computer storage media, for source data review and document compliance. The computer obtains a plurality of source documents, each source document comprising clinical trial information of the one or more clinical studies. The computer identifies, for each source document and by a natural language processing (NLP) model, a plurality of entities of the one or more clinical studies from the information related to the participants of the one or more clinical studies in the source document. The computer generates an updated NLP models configured to detect one or more events likely to have occurred among the plurality of entities, each event being associated with at least one entity from the plurality of entities. The updated NLP model is configured to update parameters in response to receiving a user input representing feedback to a model output from the updated NLP model.

### CLAIM OF PRIORITY

This application claims priority under 35 U.S.C. § 119(e) to U.S. Patent Application Ser. No. 63/582,387, filed on Sep. 13, 2023, the entire contents of which are hereby incorporated by reference.

### BACKGROUND

For a medical treatment to be adopted to treat a particular illness, clinical trials are conducted to evaluate a particular medical treatment and/or combination of treatments to treat the illness. A clinical trial and/or study is conducted to evaluate the efficacy, effectiveness, and safety of a medical treatment. Clinical trials are conducted according to protocols to ensure consistency and standardization for evaluating the medical treatment, maintaining safety of the trial or study's participants, ensuring ethical compliance, among other types of measures. A clinical trial follows protocols to provide that the clinical trial demonstrates scientific validity for the medical treatment, maintains integrity of data and other types of information collected during the trial, and complies with regulatory oversight.

### SUMMARY

The technology disclosed in this specification relates to a source data review system that applies artificial intelligence (AI) techniques to analyze source data in source documents from a clinical trial. A source document is a collection of patient data that can be found in, for example, medical records, lab reports, consent forms, prescription records, and other types of records for patient healthcare data. The AI-based source data review system (also referred to as the AI-based system) performs a process check for a clinical trial through information captured in source documents to improve protocol adherence and compliance for the clinical trial. The AI-based source data review system analyzes source data in the source documents, identifies entities in the clinical trial from the source data, and generates signals indicative of events that are likely to have occurred in association with one or more entities in the source document. The AI-based system leverages contextual data related to the one or more entities that can be found in additional source documents, e.g., other than the instant source document, and/or corpora of documents related to the clinical trial, to provide an accurate signal indicating the occurrence of an event in the clinical trial. A signal representing an event indicates an occurrence of the event associated with the one or more entities that can impact an evaluation of the clinical trial, such as a clinical trial's adherence to protocols, efficacy of the medical treatment for the clinical trial, safety of the clinical trial, among other factors. The signal can also represent a detection of non-compliant data within one or more source documents, and indicate sources of the non-compliant data, e.g., sites where source data is collected for the clinical trial. The signals generated by the AI-system can indicate the occurrence of adverse events, serious adverse events, and protocol deviations, among other types of events that can affect clinical trial outcomes.

The disclosed AI-system generates natural language processing (NLP) models that detect an event likely to have occurred among entities in the source data of the source documents. The generated NLP models for the AI-system are trained to identify entities in the source data by analyzing features of data found in a particular source document and leverages feature data from source documents that originate from different phases or different instances within a phase, of the clinical trial. The NLP models for the AI-system include neural network layers configured to generate signals indicating events using the feature data and generate model outputs indicating non-compliant data in the source document. The AI-system is configured to train and update model parameters for the NLP models to improve the accuracy of event detection. The AI-system can also be configured as a query-based recommendation engine. For example, the AI-system can receive data indicating a query related to one or more entities in any of the source documents. In response to receiving the query, the AI-system leverages NLP models trained with contextual information to generate signals detected among the entities indicated in the query. The AI system can increase a likelihood of the clinical trial meeting compliance standards, following protocols, reducing the risk of non-compliant data, increase consistency across source documents from different origination sites and phases, and provide computational efficiencies to computing platforms and devices that utilize source documents to generate clinical data.

Some approaches for source data review can include a computing device for digitizing and reviewing source documents from clinical trials for errors, but these approaches lack consistency across sites for the same clinical trial and do not provide contextual information or standardization that can be obtained by natural language processing. Furthermore, clinical data can include dense feature data from across numerous (e.g., millions) of source documents for a clinical trial and thus include several errors or inadequate oversight for clinical trial compliance. In some cases, errors can result in unreported events and symptoms that can affect patient health outcomes and undetected protocol deviations that can pose compliance risks for the clinical trial. Further still, undetected ineligibility events that result in eligible patients for the clinical trial can result in missed data correlations, e.g., concomitant medication, that reduce the efficiency of the clinical trial and inadvertent cause the clinical trial to reach an inaccurate outcome based on inaccurate clinical data. In some cases, the data found in a source document (also referred to as “source data”) can be consolidated from multiple source documents into a one document (such as a “case report form” or “CRF”) with a standardized format to collect source data for different entities to form clinical trial data. These approaches to consolidate or standardized source data from source documents do not address underlying issues within the source documents, such as non-compliant data, instances of adverse events occurring between entities in the source documents, and lack of contextual information for the entities in the source document.

The techniques described in this specification relate to a method, system, and apparatus including computer programs encoded on computer storage media, for the review and analysis of source documents in relation to the corpora of documents related to the conduct of the clinical trial and any related protocols for the clinical trial. In particular, the source data review system applies artificial intelligence techniques to generate natural language processing (NLP) models that can be queried to detect the occurrence of events in the clinical trial, with contextual information from corpora of documents related to the clinical trial to improve the accuracy of the detected signal. The disclosed techniques provide improves compliance of clinical trial data, increases the quality of clinical data generated from source data derived from the source documents, and provides an additional layer of contextual information from related corpora of documents to improve detection accuracy of the events. In this way, the disclosed technology can provide additional context for fields, formats, and other aspects of source data that may not have been previously identified otherwise without applying the NLP models to perform source data review.

The system disclosed in this specification can improve clinical trial quality by enabling consistent reporting of adverse events, end-of-study/treatment, and adherence to regulatory requirements, thereby reducing risks and allowing for timely trial completion. The system can reduce the time taken for source data review thereby enabling early detection of signals from data. The system can detect recruitment errors before trials reach a particular phase or for a particular patient, e.g., contradictory medication or medical history was not reported. The AI-based system disclosed technology can also improve compliance among clinical trial sites by flagging and updating non-compliance data in clinical trial documents. For example, the AI-based system can determine the compliance status of source documents and reduce document review cycle time, e.g., processing associated with data transmission of source documents between different instances and/or stages of the clinical trial.

By reducing document review cycle time, any computing systems, platforms, and devices for the clinical trial can achieve a timely database lock (DBL), reducing time to lock out databases after collecting clinical data and improve timeliness in clinical study closeout. Timeliness in database locking and closing out clinical studies can reduce potential delays in analyzing the results and performing statistical testing that leverages the clinical trial data for medical discovery, studies, etc. The NLP models can be trained to identify patterns of defects that indicate non-compliance in source documents and other corpora of documents can be trained to perform inference tasks, e.g., identifying outliers that can result in non-compliant data. The identified patterns can be provided as a visualization to clinicians of the clinical trial, to flag sources of non-compliance more readily in a clinical study, particular those that can affect the validity of the study. Reducing sources of error in clinical studies can remove impediments to medical research and efficacy studies, e.g., such as those performed when testing medications. As an example, clinical sites at certain countries and/or regions of a country can have more defects than average defect rate, e.g., due to lack of oversight and monitoring. Furthermore, identifying of documentation non-compliance at earlier stages of clinical trials and studies can provide early detection of potential quality and compliance issues.

The application of artificial intelligence techniques by the system to generate NLP models can improve detectability of errors in the content entered into documents and the detectability of potential compliance issues, based on a holistic analysis of related corpuses of documents. Examples of content errors in documents can include using patient names instead of an identification number, while examples of compliance issues can include a clinical rule not being followed when performing the clinical study. The application of the near real-time communication and feedback based on the detected errors can provide a timely notification of the detected errors to teams of clinicians at clinical sites. This can reduce the time needed to take corrective actions, compared to approaches that independently review and analyze scanned documents.

The disclosed technology improves the efficiency of clinical studies and improves quality of studies by allowing consistent reporting of adverse events, end-of-study, treatment throughout the clinical trial, and adherence to regulatory requirements for the clinical trial. Thus, the disclosed system can reduce risks of delay of clinical trials and increase rates of on-time completion of the clinical trials.

In one general aspect, a method includes: obtaining, from one or more data sources for one or more clinical studies, a plurality of source documents. Each source document from the plurality of source documents includes clinical trial information of the one or more clinical studies. The method includes identifying, for each source document in the plurality of source documents and by a natural language processing (NLP) model, a plurality of entities of the one or more clinical studies from the information related to participants of the one or more clinical studies in the source document. The NLP model can be trained to identify the plurality of entities by analyzing feature data of (i) the information related to the participants of the one or more clinical studies across the plurality of source documents, and (ii) one or more corpora of documents for the clinical trial related to the plurality of source documents. The method includes generating, based on the plurality of entities and using the analyzed feature data, an updated NLP model including a plurality of layers and configured to detect one or more events likely to have occurred among the plurality of entities. Each event from the one or more events can be associated with at least one entity from the plurality of entities. The updated NLP model can be trained using the analyzed feature data from at least a subset of the plurality of source documents and using a subset of one or more corpora of documents for the clinical trial as contextual data for the at least one entity. The updated NLP model is configured to update one or more parameters of at least one layer from the plurality of layers in response to receiving a user input representing feedback to a model output from the updated NLP model.

Other embodiments of this and other aspects of the disclosure include corresponding systems, apparatus, and computer programs, configured to perform the actions of the methods, encoded on computer storage devices. A system of one or more computers can be configured by virtue of software, firmware, hardware, or a combination of them installed on the system that in operation cause the system to perform the actions. One or more computer programs can be so configured by virtue having instructions that, when executed by data processing apparatus, cause the apparatus to perform the actions.

The foregoing and other embodiments can each optionally include one or more of the following features, alone or in combination. For example, one embodiment includes all the following features in combination.

In some implementations, the method includes receiving, from a computing device communicatively coupled to the one or more computers, an input query related to at least one entity from the plurality of entities, and generating, based on the input query and by the updated NLP model, a signal representing one or more events associated with the at least one entity.

In some implementations, at least one event from the one or more events detected by the updated NLP model indicates a correlation between two or more entities from the plurality of entities.

In some implementations, the clinical trial information includes at least one of (i) data related to participants, (ii) data related to clinicians, (iii) data related to study protocols, or (iv) data related to regulations, for the one or more clinical studies.

In some implementations, generating the updated NLP model includes generating, for each source document in the plurality of source documents, a classification of each respective entity from the plurality of entities. The classification can indicate a class of medical ontology for the respective entity based on the analyzed feature data.

In some implementations, the updated NLP model is configured to generate a plurality of events likely to have occurred among the plurality of entities. The updated NLP model can be configured to generate, for each event in the plurality of events, a value indicating a likelihood of association/correlation of entities from at least a subset of the plurality of entities.

In some implementations, training the updated NLP model includes providing a training example query for input to the updated NLP model, and generating, using the training example query and by the updated NLP model, a training model output representing one or more detected events associated with plurality of entities. Training the updated NLP model can include obtaining ground truth data. The ground truth data can indicate one or more events associated with the plurality of entities, and determining a score based on a comparison of the ground truth data and the training model output. Training the updated NLP model can include, based on the score exceeding a threshold, updating one or more parameters of at least one layer from the plurality of layers.

In some implementations, the method includes detecting, using the updated NLP engine, an adverse event from the plurality of events. The adverse event can indicate that the information related to the participants does not follow a protocol from the one or more protocols for conducting the one or more clinical studies. The method can include, in response to detecting the adverse event, generating data indicative of one or more updates to the information related to the participants found in the source document from the plurality of source documents that includes an entity associated with the adverse event.

In some implementations, the method includes generating, by the updated NLP model, generative prompt data that configures a user interface of a client device. The generative prompt data causes display of a visual representation of annotations corresponding to each respective entity from the plurality of entities. Each annotation can indicate the class of medical ontology for the respective entity. The NLP model can be trained to generate the generative prompt data using one or more generative visualization techniques.

In some implementations, the method includes providing the generative prompt data to the client device. Providing the generative prompt data causes the client device to update the user interface to include one or more graphical elements, each graphical element corresponding to each annotation from the annotations.

In some implementations, the method includes providing, for output by the one or more computers, the user interface including a respective selectable control for providing feedback to an identification of an event from the one or more events for the one or more clinical studies, the identified event corresponding to a graphical element from the one or more graphical elements. The method can include receiving, by the user interface, a user selection of one or more of the selectable controls included in the user interface and updating, one or more parameters of the plurality of layers for the updated NLP model.

In some implementations, the method includes determining, from the plurality of events and using the NLP model, one or more instances of non-compliant data in at least one source document from the plurality of source documents. The non-compliant data can be associated with an entity from the plurality of entities.

In some implementations, an instance from the one or more instances of non-compliance comprises a deviation from at least one protocol from one or more protocols for conducting the one or more clinical studies.

In some implementations, an instance from the one or more instances of non-compliant data indicates a treatment plan that does not follow protocol for the one or more clinical studies.

In some implementations, the method includes identifying, based one or more instances of non-compliant data, an output trend indicating a pattern of non-compliance for the one or more clinical studies, the pattern being associated with at least one (i) a subset of entities from the plurality of entities, or (ii) one or more sites for conducting the one or more clinical studies.

In some implementations, the method includes monitoring, by the updated NLP models, input data from a computing device communicatively coupled to the one or more computer systems. The method can include detecting, from the input data and by the updated NLP models, a detection of non-compliant data in the input data. The method can include generating, based on the detection of the non-compliant data and using the updated NLP models, one or more of (i) a signal indicating the detection of the non-compliant data, or (ii) at least one adjustment for the non-compliant data.

In some implementations, the method includes obtaining one or more documents corresponding to one or more sites for conducting the one or more clinical studies, the one or more documents comprising clinical data for the one or more clinical studies. The method can include determining, based on the one or more documents, a plurality of data fields and a plurality of data formats for the clinical data from the one or more documents and identifying, by the updated NLP model and based on the one or more documents, at least corpus of documents from a subset of the one or more corpora of documents related to the one or more documents. The method can include applying, by the updated NLP model, a set of compliance rules to the clinical data for the one or more documents. Applying the set of compliance rules can include identifying one or more instances of non-compliant data in the clinical data from the one or more documents, generating, based on the one or more instances of non-compliant data in the clinical data and a set of quality indicators for the clinical data, a score representing a compliance rating for the one or more documents, and generating, based on the one or more instances of non-compliant data, a signal indicating one or more fields in at least one document from the one or more documents that include at least one instance from the one or more instances of non-compliant data. The method can include providing at least one of (i) the score for the compliance rating for the at least one document, or (ii) the signal indicating the one or more fields in the at least one document, to a computing device.

In some implementations, the one or more documents can include at least one of (i) certification records, (ii) delegation tasks, (iii) training logs, (iv) financial disclosures, or (v) a set of protocols.

In some implementations, the updated NLP model is configured to identify a trend from the one or more instances of non-compliant data in the clinical data, the trend indicating the one or more documents that do not meet at least one protocol from the one or more protocols or at least one rule in the set of compliance rules.

In some implementations, the method includes determining, by the updated NLP model and based on the set of compliance rules, a non-compliance rate of a set of documents, the set of documents associated with a site from the one or more sites and a threshold value for non-compliant data for the site. The method can include comparing the non-compliance rate to a threshold value for non-compliant data for the site and based on the non-compliance rate to the threshold value, providing the signal indicating the one or more instances of non-compliant data in the set of documents to a computing device.

In some implementations, the method includes analyzing, by the updated NLP model, the one or more documents. Analyzing the source documents can include comparing one or more site fields in the one or more documents to one or more fields in the one or more corpora of documents. The method can include, based on the comparison, generating a set of indicators for a set of fields, each indicator in the set of indicators corresponding to a field in the set of fields. The indicator from the set of indicators for the field in the set of fields represents compliance status of data represented by the field.

In one general aspect, a source document review system includes a computing device comprising at least one processor and a memory communicatively coupled to the at least one processor, the memory storing instructions which, when executed by the at least one processor, cause the at least one processor to perform operations. The operations include obtaining, from one or more data sources for one or more clinical studies, a plurality of source documents. Each source document from the plurality of source documents includes clinical trial information of the one or more clinical studies. The operations include identifying, for each source document in the plurality of source documents and by a natural language processing (NLP) model, a plurality of entities of the one or more clinical studies from the information related to participants of the one or more clinical studies in the source document. The NLP model can be trained to identify the plurality of entities by analyzing feature data of (i) the information related to the participants of the one or more clinical studies across the plurality of source documents, and (ii) one or more corpora of documents for the clinical trial related to the plurality of source documents. The operations include generating, based on the plurality of entities and using the analyzed feature data, an updated NLP model including a plurality of layers and configured to detect one or more events likely to have occurred among the plurality of entities. Each event from the one or more events can be associated with at least one entity from the plurality of entities. The updated NLP model can be trained using the analyzed feature data from at least a subset of the plurality of source documents and using a subset of one or more corpora of documents for the clinical trial as contextual data for the at least one entity. The updated NLP model can be configured to update one or more parameters of at least one layer from the plurality of layers in response to receiving a user input representing feedback to a model output from the updated NLP model.

In one general aspect, a non-transitory computer-readable storage device storing instructions that when executed by one or more processors of a computing device cause the one or more processors to perform operations. The operations include obtaining, from one or more data sources for one or more clinical studies, a plurality of source documents. Each source document from the plurality of source documents includes clinical trial information of the one or more clinical studies. The operations include identifying, for each source document in the plurality of source documents and by a natural language processing (NLP) model, a plurality of entities of the one or more clinical studies from the information related to participants of the one or more clinical studies in the source document. The NLP model can be trained to identify the plurality of entities by analyzing feature data of (i) the information related to the participants of the one or more clinical studies across the plurality of source documents, and (ii) one or more corpora of documents for the clinical trial related to the plurality of source documents. The operations include generating, based on the plurality of entities and using the analyzed feature data, an updated NLP model including a plurality of layers and configured to detect one or more events likely to have occurred among the plurality of entities. Each event from the one or more events can be associated with at least one entity from the plurality of entities. The updated NLP model can be trained using the analyzed feature data from at least a subset of the plurality of source documents and using a subset of one or more corpora of documents for the clinical trial as contextual data for the at least one entity. The updated NLP model can be configured to update one or more parameters of at least one layer from the plurality of layers in response to receiving a user input representing feedback to a model output from the updated NLP model.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A is a block diagram that illustrates an example of a system for source document review using artificial intelligence techniques.

FIG. 1B is a block diagram that illustrates another example of a system for source document review using artificial intelligence techniques.

FIG. 1C is a block diagram that illustrates an example model output of the system for source document review using artificial intelligence techniques.

FIG. 1D is a block diagram that illustrates another example model output of the system for source document review using artificial intelligence techniques.

FIG. 2A is a diagram that illustrates an example input for the source document review system of FIGS. 1A and 1B.

FIG. 2B is a diagram that illustrates an example output of the system of FIGS. 1A and 1B.

FIG. 2C is a diagram that illustrates another example output of the system of FIGS. 1A and 1B.

FIG. 2D is a diagram that illustrates another example output of the systems of FIGS. 1A and 1B.

FIG. 3A is a flow diagram that illustrates an example of a process for detecting events that have occurred between entities of source documents for a clinical study.

FIG. 3B is a flow diagram that illustrates an example of a process for generating signals indicating non-compliant data in source documents.

FIG. 4 shows a block diagram of a computing system.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

A clinical trial, including any clinical studies that makeup the clinical trial, relies on source documents to capture medical data such as diagnoses, prescription dosages, treatment history, and other types of information related to entities in the trial. A source document is a collection of raw data for an entity in a clinical trial, and can include medical records, health records, laboratory reports, consent forms, and other types of records or documents pertaining to an entity, e.g., a participant, in the clinical trial. Examples of an entities can include medical conditions, diagnoses, prognoses, treatment events, dosages, patient information, clinician information, among other types of entities in a source document.

An inconsistency, error, or any form of data quality degradation can generate inaccurate evaluations for a clinical trial. Inaccurate clinical trials evaluations can result in a particular treatment failing protocols due to poor data integrity, rather than non-compliance to healthcare protocols and thus prevent preventing access to a medication that would otherwise improve health outcomes. As another example, an inaccurate trial evaluation inadvertently allow access to a medication that would be detrimental to patient health outcomes by seemingly achieving protocols but having inconsistencies in the source data.

Source data can also have contextual information obtained from other corpora of documents related to the clinical trial, such as trial documents and guidelines, critical data processes, ontology databases, among others. Examples of contextual data for a source document can include patient demographics, treatment information, clinical setting, timing of data collection, patient conditions, measurement methods, protocol adherence, source document origin, healthcare provider documentation and notes, and external factors, e.g., changes in healthcare provider/setting policies, equipment malfunction, and environmental factors. Thus, different source documents within a single phase of a clinical trial, and/or across different phases of a clinical trial, can capture different contextual information (from other source documents, and/or corpora of additional documents for the clinical trial) for one or more entities.

The disclosed technology is an AI-based system that can identify entities from medical records, e.g., medical condition, diagnosis, medication, and perform steps of source data review to ensure patient safety, adherence to clinical trial protocol and improve quality of care throughout the clinical trial. The AI-based system identifies entities in the source data by applying digitization and text analytics techniques to source documents, which are often handwritten forms or images of handwritten text. The AI-based system determines an association with entities in the source data to terms in medical ontologies to generate an NLP model. The NLP model is trained to detect occurrence of events among one or more of the identified entities. The AI-based system can generate training sets of generative prompts to evaluate information in source data, and train using the generative prompts to identify correlation in source data from the source documents, e.g., to identify patterns of non-compliance from the source documents. The AI-based system can also generate visual representations of source data with annotations to classify entities using medical ontologies, flag data, e.g., missing, inaccuracy, or inconsistent, among other types of non-compliant data. The AI-based system can also be configured to detect and redact personal identifiable information to reduce the risk of data privacy violations in a clinical trial.

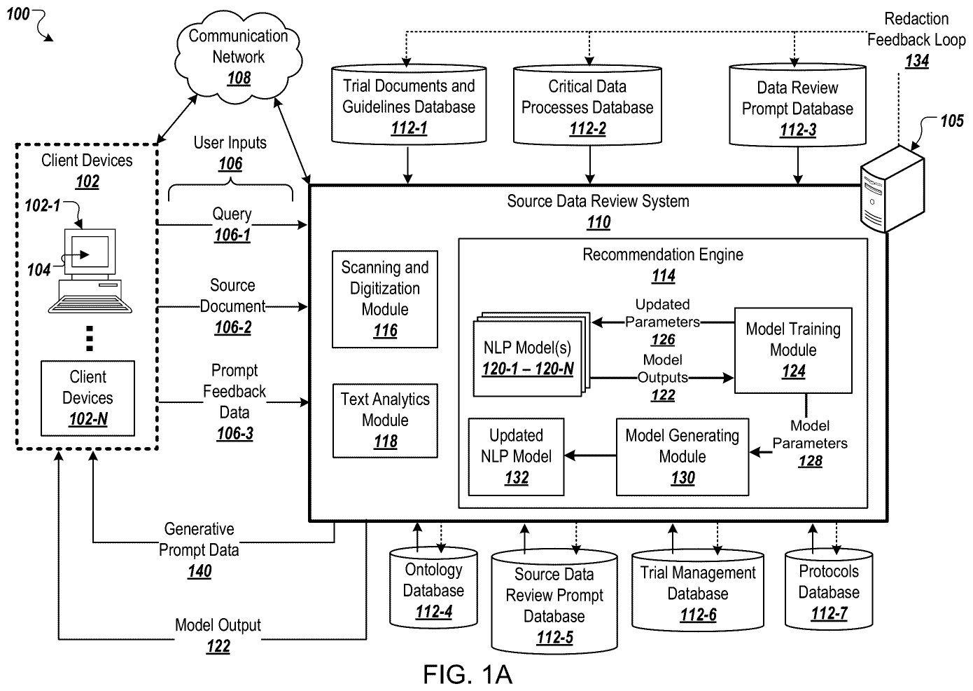

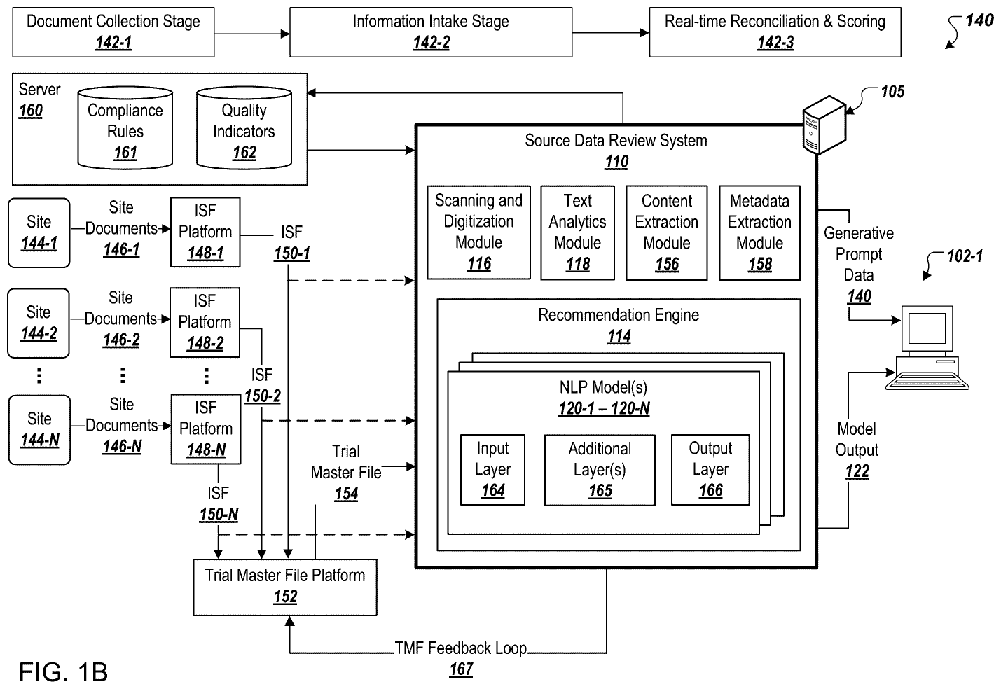

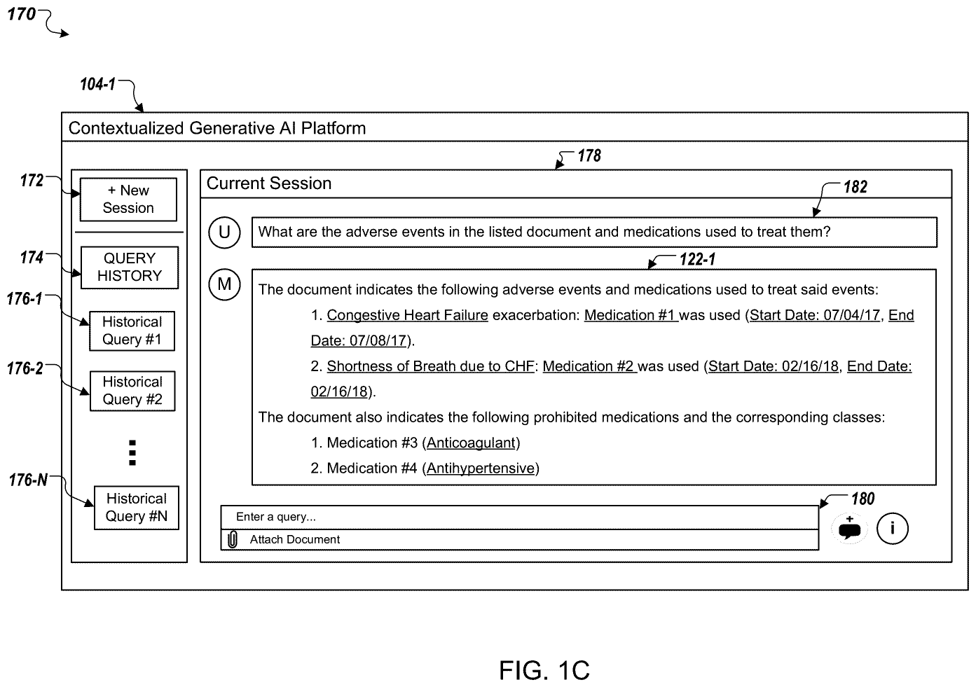

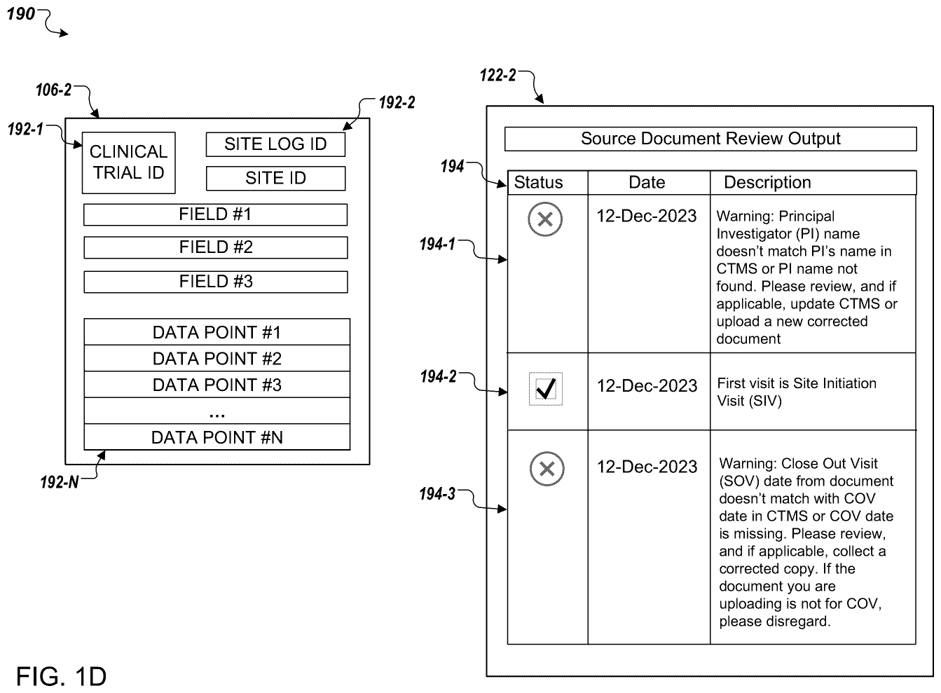

FIG. 1A is a block diagram 100 that illustrates an example of a system for source document review using artificial intelligence techniques. Block diagram 100 shows an example source data review system 110 (also referred to as a “system 110”) configured to applying AI techniques to source documents. The system 110 can also be referred to as a “source document review system 110.” A source document can include a form of clinical trial information, e.g., data related to patients, participants, clinicians, protocols, and other related data that is collected for clinical studies in a clinical trial.

As illustrated in FIG. 1A, the system 110 is deployed on a computing device 105, but can deployed on other types of computing devices, systems, platforms, etc., that can be communicatively coupled to other computing hardware, e.g., by communication network 108. Examples of communication network 108 can include Bluetooth, Wi-Fi, the Internet, etc.

The system 110 includes a recommendation engine 114 (also referred to as “engine 114”) configured to generate a number of natural language processing (NLP) models 120-1 through 120-N (collectively “NLP models 120”). The engine 114 of the system 110 generates the NLP models 120 by analyzing corpora of documents related to the clinical trial from a number of databases 112-1 through 112-7 (collectively “databases 112”).

Although FIG. 1A illustrates databases 112-1 through 112-7, the system 110 can be configured to obtain source documents and other corpora of documents from any number databases. A corpus of documents obtained from the databases 112 can be an example additional documents related to clinical trials, which can be used as inputs for the system 110 to generate NLP models 120 for source data review. As an example, database 112-1 is illustrated in FIG. 1A as trial documents and guidelines database 112-1, which can include corpora of clinical trial and/or guideline documents. Examples of clinical trial documents can include study protocols, informed consent forms, monitoring reports, audit reports, regulatory approvals, data management plans, among other types of clinical trial documents. Examples of clinical guideline documents can include clinical practice standards, technical trial design, safety, and reporting requirements, ethical guidelines, standard operation procedures, regulator guidelines, etc.

In some cases, one or more of the databases 112 can store of model outputs 122 and/or generative prompt data 140 as a historical output, e.g., for training the NLP models 120. As another example, database 112-5 is illustrated in FIG. 1A as source data review prompt database 112-5, which can include historical model outputs from current and/or previously generated NLP models 120 for the system 110. In some cases, source documents for the system 110 can be obtained (e.g., wirelessly and/or through a wired connection) from one or more databases from the databases 112.

In addition to the trial documents and guidelines database 112-1 and source data review prompt database 112-5, FIG. 1A depicts a critical data processes database 112-2, a data review prompt database 112-3, an ontology database 112-4, a trial management database 112-6, and a protocols database 112-7. The critical data processes database 112-2 can be a database that stores a corpus of documents related to rules for conducting the clinical trial, which can include regulatory and compliance guidelines. The data review prompt database 112-3 can be a database that stores historical and current user inputs 106 provided to the system 110 and/or other instances of the system 110, e.g., in the case of the system 110 configured as a distributed computing platform across multiple computing devices. The ontology database 112-4 can be a database of medical information and terminology, including other contextual terms and related information for a particular word in the medical field. The trial management database 112-6 can be a database with a corpus of documents for maintaining data related to activities in the clinical trial. The protocols database 112-7 can be a database with a corpus of documents for regulating data related to activities in the clinical trial according to design parameters for the clinical trial, e.g., to increase the likelihood of a successful, accurate evaluation of the medical treatment being evaluated in the clinical trial. In some implementations, a database from databases 112 can include training examples for generating and updating the NLP models 120.

The NLP models 120 can be generated from corpora of documents retrieved from the databases 112. One or more corpora of documents from the databases 112 can be pre-processed by the engine 114, which can include obtaining data each document in the corpora of documents and generating tokens to represent words, numbers, and other characters, e.g., punctuation marks, symbols. By generating tokens for information found in the corpora of documents, the engine 114 can provide an input data structure in a format that is more readily analyzed by an NLP model, e.g., allowing the NLP model to perform a semantic analysis and identify entities in a document. The engine 114 can also be configured to other pre-process data from documents to improve the consistency of data inputs for generating the NLP models, such as by removing stop words, modifying/converting words to different forms or representations, etc.

By using multiple types and instances of document corpora, the engine 114 is configured to generate NLP models that are particularly trained to learn contextual information for entities using contextual data in the corpora of documents, e.g., learning trends and patterns from disparate documents and data sources that can be applied to entities in the source document. In some implementations, the corpora of documents from the databases 112 can include patient data such as medical history, adverse events, current medication, among other examples of patient data. In some implementations, the corpora of documents can include clinical study documents, guidelines, and other types of documents associated with conducting a clinical trial.

The engine 114 can be configured to apply a number of statistical techniques to build training and testing sets for NLP model generation by generating data structures representing features of the text found the multiple corpora of documents retrieved by the system 110. For example, the engine 114 can apply statistical techniques such as Bag of Words (BoW) and/or Term Frequency-Inverse Document Frequency (TF-IDF) to convert words into numerical feature data. The engine 114 can also be configured to generate feature data by applying machine learning techniques that embed text from the source documents into feature data. In some implementations, the engine 114 is configured to generate word embeddings from the tokens representing data in corpora of documents. As another example, the engine 114 can be configured to generate contextual embeddings for text in a particular corpus of documents, based on contextual information from another one or more corpora of documents different than the particular corpus of documents.

Using the feature data as an input, the engine 114 is configured to apply a number of machine learning techniques and/or deep learning approaches to determine model parameters for the NLP models 120. Examples of the machine learning techniques can include Support Vector Machines (SVM), Random Forest, decisions trees, etc. In some implementations, the engine 114 can be configured to apply deep learning techniques, e.g., including neural networks, to generate model parameters for generating the NLP models 120. In some cases, this can include applying machine learning techniques to determine model parameters for the layers of the NLP models 120. As described in reference to FIG. 1B below, an NLP model can be generated using model parameters from the machine learning techniques to generate the layers of the NLP model. In some implementations, other types of machine learning techniques such as transformers can be used to generate the NLP models.

In some implementations, the engine 114 is configured to apply retrieval-augmented generation techniques to generate, update, and/or train the NLP models and/or an updated NLP model 132 using the additional corpora of documents as contextual data for training the models. By applying retrieval-augmented generation techniques, the resulting NLP models can be trained to generate model outputs that leverage contextual data and provide an output with higher accuracy than an output without leveraging the contextual data. In some implementations, the engine 114 is configured to apply similarity matching between terms in an input document to the corpora of documents from databases 112 and leverages the additional context from the corpora of documents to generate a model output for the input document.

Referring to the system 110, a number of client devices 102-1 through 102-N (collectively “client devices 102”) can be communicatively coupled to the system 110 by the communication network 108. As an example, FIG. 1A depicts a client device 102-1 as a computing device with a display 104, e.g., for presenting or displaying data.

The system 110 can be configured to receive any number of user inputs 106. The system 110 is configured to generate, based on the user inputs 106 and using the NLP models 120, one or both of a model output 122 and generative prompt data 140. A model output 122 can be a signal generated by one or more of the NLP models 120, the signal indicating an occurrence of one or more events related to entities in the user inputs 106. In some implementations, the signal can be referred to as a detection of the one or more events but may also be referred to as an inference of one or more events related to the entities. The NLP models 120 can be configured to generate the signal representing the event for the entities indicated by the user inputs, by training the NLP models using corpora of documents obtained from one or more of databases 112. This is because the corpora of documents can provide additional contextual information related to the entities from varying types of documents that may not be found in a source document that includes one or more of the same entities. The NLP models 120 can also be configured to generate and transmit generative prompt data 140 as an output, e.g., to a client device from client devices 102. The generative prompt data can include data that configures the user interface of a client device to provide a response to a user input. For example, generative prompt data 140 can include a response to an input query, providing text describing the detected events associated with entities in the input query.

In some implementations, the generative prompt data 140 can include of visualization of data related to the user input. The visualization of data can include a digital copy of an annotated source document, with annotations indicating different entities according to a medical ontology and/or class of ontology for the entity. The system 110 can provide the generative prompt data 140 to a client device, thereby causing the client device to update a user interface and display the user interface with one or more graphical elements, e.g., indicating an annotation and/or any type of response to a user input. In some implementations, an annotation can indicate an instance of missing data, inconsistent data, a trend or pattern in the data, or a detected event, for one or more entities in the source document.

The user inputs 106 can include an input query 106-1 received from a client device 102, in which the input query 106-1 relates to a request to detect events for one or more entities in a clinical trial, e.g., an entity in a source document or corpora of documents related to the clinical trial. Alternatively or in addition to the input query 106-1, the user inputs 106 can include a source document 106-2 as an input to the system 110 relating to a request to analyze the source document. The request for the source document 106-2 can include a request for identifying and optionally, classifying entities in the source document. The system 110 can also be configured to generate detections indicating events involving entities found in the source data of the source document 106-2. In some implementations, the source document 106-2 is accompanied by a query 106-1 as an input to the system 110, and the system 110 can generate signals for entities in the source document that are included in the query 106-1.

The model output 122 can be a signal generated by the NLP models 120 indicating an event that is likely to have occurred between one or more entities from user inputs 106. An entity can be identified from user inputs 106, such as the reference to an entity in a query 106-1 and/or a source document, and one or more events can be detected by the NLP models 120 in the form of model output 122. Examples of model output 122 can include one or more entities being associated with a detected event, including adverse events, serious adverse events, efficacy events, clinical endpoints, protocol deviation events, dropout/withdrawal events, recruitment events, and endpoint events, among others. For example, an adverse event indicates the one or more entities of a source document and/or an input query being associated to an instance of a side effect, e.g., unintentional symptom, associated with the medical treatment, whereas a serious adverse event indicates an instance of an adverse event that can result in death, hospitalization, among other potentially dangerous effects of the medical treatment. As another example, a protocol deviation event for the one or more entities indicates an instance where conducting the clinical trial can deviate from the clinical trial's protocols, e.g., design parameters, regulations, and other factors, that can impact the outcome of the clinical trial.

In some implementations, the NLP models 120 can be large language models (LLMs) trained using the one or more corpora of documents from databases 112. In some implementations, the model output 122 can be a signal indicating a deviation from a previously detected event, e.g., a change in the severity of an event.

In some implementations, an event can include a correlation between entities indicated in a user input. The correlation can indicate a detected relationship between the two entities, linking the two entities for any clinical data generated for the clinical trial. In some cases, the correlation can be represented by a value indicating a likelihood of the event having occurred among entities. In some implementations, multiple model outputs can be generated, each model output having a likelihood of a corresponding event likely to have occurred between the entities. The model outputs can be ordered, e.g., ranked from highest likelihood to lowest likelihood, by the system 110. For example, the system provides signals indicative of events with a higher likelihood prior to providing signals indicative of events with a lower likelihood.

The system 110 includes a scanning and digitization module 116 and a text analytics module 118 to pre-process data from the user inputs 106 into a digital data format for processing and analysis by the engine 114. In some cases, the system 110 processes one or more corpora of documents from the databases 112 using the scanning and digitization module 116 and/or the text analytics module 118, e.g., for generating the NLP models 120. For example, the scanning and digitization module 116 is configured to scan and digitize information found in a user input 106 that includes a source document 106-2. Because a source document containing source data is can often be a handwritten document, a source document 106-2 is likely to be an image of a handwritten document. The scanning and digitization module 116 can be configured to apply a number of optical character recognition (OCR) techniques to convert the source data in the source document 106-2 into digital data formats, e.g., for feature engineering and analysis by the recommendation engine. As another example, the text analytics module 118 is configured to apply computational techniques to convert unstructured source data in the source document to generated structured source data. In some implementations, the text analytics module 118 is configured to extract features of the data found in documents provided to the system 110 and generates feature vectors to represent the feature data.

In some implementations, the user inputs 106 can include prompt feedback data 106-3 from a client device. The prompt feedback data 106-3 can be provided from a client device in response to receiving generative prompt data 140 from the system 110. For example, the system 110 can generate NLP models 120 and receive a user input 106 indicating one or both a query 106-1 and a source document 106-2. The system 110 can generate, in response to receiving the user input 106, generative prompt data 140 (instead of, or in addition to the model output 122) that provides a selectable control on a graphical user interface (GUI), e.g., by providing data that configures user interface elements of the client device 102, to allow the user to confirm or reject events detected by the NLP models 120. The prompt feedback data 106-3 can be user feedback to the generative prompt data 140, providing a response to the generative prompt data 140 (and/or the model output 122 in cases where the model output 122 is provided). The prompt feedback data 106-3 can be provided to engine 114 as input for updating the NLP models 120, e.g., for model training.

In some implementations, the NLP models 120 can determine an occurrence of non-compliant data that can include the display or inclusion of data for an entity, in which the inclusion of said data does not follow a particular clinical trial guideline. Compliance of document can refer to the compliancy of presenting information in a document according to regulatory guidelines. For example, the NLP models 120 can identify an event indicating personal identifiable information (PII) for a participant that is not redacted in a source document, for a scenario in which the PII may be redacted so that the clinical trial can proceed in accordance with one or more guidelines, protocols, etc. The system 110 can apply a redaction feedback loop 134 identify one or more instances of the non-compliant data in one or more of the databases 112 to redact the non-compliant data, e.g., in a corpus of documents for the database.

The engine 114 includes a model training module 124 configured to train NLP models 120 by obtaining a set of model outputs 122 and generating updated parameters 126 based on an analysis of the model outputs 122. In some cases, the model training module 124 us configured to generate model parameters for the generation of an updated NLP model that is different than any of the NLP models 120. For example, the model training module 124 can generate model parameters 128 based on the model outputs 122 of the NLP models 120 and provides the model parameters 128 to a model generating module 130. The model generating module 130 is configured to generate an updated NLP model 132 according to the model parameters 128.

Training of any of the NLP models, e.g., NLP models 120, updated NLP model 132, can be performed using obtained ground truth data that includes known labels, associations, classifications, etc., coupled with a corresponding input, e.g., some or all of the entities in the source data, some or all source data related to entities found in corpora of documents, or some combination thereof. The model training module 124 is configured to adjust one or more weights or parameters of the NLP models 120 to match signals from the ground truth data. In some implementations, a model from the NLP models and/or the updated NLP model 132 includes one or more fully or partially connected layers. Each of the layers can include one or more parameter values indicating an output of the layers. The layers of the model can generate outputs for which the model can use for performing one or more inference tasks. The models can be validated and tuned through holdout and test techniques, model comparison, and model selection.

Any of the models depicted in FIG. 1A can be trained by the system 110 using a variety of training techniques, e.g., performed by the model training module, to improve the accuracy of inference tasks performed by the respective model. These training techniques can include supervised and unsupervised learning. The models can include any form of boosting techniques such as gradient boosting but can include deep learning techniques for to perform an inference task. For example, an inference task for an NLP model and/or an LLM can include performing text classification, entity identification, machine translation, and other types of semantic analysis. In some examples, a model performs hybrid-learning techniques to improve accuracy of model output. Training processes for the models depicted in FIG. 1A can include any number of iterative processes, each performing iterations to train the model to achieve a target performance value, e.g., an error rate below a threshold value, a generated classification label that matches the ground truth label.

In some implementations, a component of the system 110 is coupled to some or all of the other components of the system 110 (e.g., the scanning and digitization module 116, the text analytics module 114, the recommendation engine 114, the model training module 124, the model generating module 130), by a wired connection, wireless connection, etc.

Referring to FIGS. 1A and 1B, the system 110 can be communicatively coupled to the one or more client devices 102, e.g., by a communication network 108. The system 110 can be configured to monitor data within a client device 102-1, and/or data transmitted between the client device 102-1 and the system 110. The system 110 can generate a signal, e.g., a detection, indicating an event likely to occur as the data is inputted into the client device 102-1 and/or transmitted from the client device 102-1 to the system 110. In this way, the system 110 can be configured to detect the occurrence of an event in the user input and in response to detecting the user input, generate one or more model outputs representing a correction to adjust input data. By providing a pre-emptive correction to the input data, the system 110 can prevent entry of non-compliant data from being stored, e.g., within a client device 102-1, and transmitted, e.g., to computing devices communicatively coupled to the client device 102-1. In some cases, electronic source documents can be presented on a client device 102-1 and data can be input, e.g., by a user of the client device 102-1, into fields of the electronic source document. The system 110 can be configured to detect instances of non-compliance in the fields of electronic source document and prevent transmission of non-compliant data in electronic source documents by providing signals indicating corrections to the non-compliant data that would allow the data to meet protocols for the clinical trials.

For example, the system 110 can monitor entry of data such as the user inputs 106 prior to the transmission of data for the user input 106. A user input 106 can include patient health data, such as a number of blood pressure measurements collected for a patient during a phase of a clinical trial. The system 110 can be configured to detect the occurrence of an event in the user input 106, such as an insufficient amount of data collected to meet a protocol for the phase of the clinical trial. The system 110 can also provide a model output 122 that includes signals to indicate an occurrence of the detected event, e.g., insufficient and/or inaccurate data collected. The model outputs 122 can also include signals to provide one or more instructions, e.g., for display 104 of the client device 102-1, to indicate and/or apply corrections to an instance of the detected event. For example, the system 110 provides a signal of non-compliant data prior to the transmission and/or storage of non-compliant data. The system 110 can provide a signal indicating one or more adjustments to modify the non-compliant data to be compliant.

FIG. 1B is a block diagram 140 that illustrates another example of the system 110 performing source document review using artificial intelligence techniques. FIG. 1B illustrates a number of stages for the system 110, including a document collection stage 142-1, an information intake stage 142-2, and a real-time reconciliation and scoring stage 142-3.

In the document collection stage 142-1, FIG. 1B shows a number of investigator sites 144-1 through 144-N (collectively “investigator sites 144”) providing sets of site documents 146-1 through 146-N (collectively “site documents 146”) to a number of corresponding investigator site file platforms 148-1 through 148-N (collectively “ISF platforms 148”). The investigator sites 144 are associated with a clinical trial and can be referred to a clinical trial sites for a clinical trial. Each site generates a set of site documents capturing data at the clinical trial site, which can be collected by healthcare practitioners, clinicians, and other types of staff at the site. Examples of site documents 146 can include informed consent forms, records of site staff, training, regulatory approvals, among other types of information collected clinical trial sites. a set of corresponding site documents through an investigator site file (ISF) platform 150.

The ISF platform 150 for a site is configured to generate an investigator site file that represents all the data captured by different types of site documents for a particular clinical trial site. Each ISF platform 150 can be configured to transmit a respective ISF to a trial master file (TMF) platform 152, which is configured to generate a trial master file 154 that represents consolidated data collected across different ISFs 150 for different clinical trial sites 144. While an ISF 150 can include documents related to the conduct of a trial at a particular clinical trial site, the TMF 154 can be a collection of data representing a corpus of documents for the entirety of the clinical trial. Each of the platforms, such as the ISF platforms 148 and the TMF platforms 152, can include one or more computing devices, networks, and other related computer hardware.

In the implementation depicted in FIG. 1B, the system 110 is configured to process and analyze a trial master file 154 as a user input, e.g., user input 106. For example, the system 110 can be configured to generate and/or update NLP models 120 to handle queries and/or detect events that are associated with entities found in the TMF 154. At the information intake stage 142-2, the system 110 obtains the TMF 154 and can process data in the TMF using the scanning and digitization module 116 and/or the text analytics module 118. In some implementations, the system 110 includes a content extraction module 156 and a metadata extraction module 158. The context extraction module 156 can be configured to determine clinical data relating to the clinical studies of the clinical trial, while the metadata extraction module 158 is configured to determine data fields and formats for the TMF 154.

In the real-time reconciliation stage and scoring stage 142-3, the system 110 can be configured to generate the NLP models 120 using the approaches described in reference to FIG. 1A above. Although FIG. 1B shows event detection and signal generation using the TMF 154 as an input, the same approach can be applied to source documents described in reference to FIG. 1A above. As another example, the dashed lines illustrated in FIG. 1B extending from the ISF platforms 148 to the system 110 indicated that the ISF 150 for a site 144 can be provided for input to the system 110.

In contrast to FIG. 1A, FIG. 1B depicts the system 110 being communicatively coupled to a server 160 configured to store compliance rules 161 and quality indicators 162 for a clinical trial. Compliance rules 161 can include guidelines and/or regulations for the clinical trial to follow, e.g., legal standards, ethical standards. The engine 114 can be configured to apply, using the NLP models 120 and one or more corpora of documents, the compliance rules 161 to the TMF 154 to identify events that indicate instances of non-compliant data in clinical data captured by the TMF 154. The engine 114 can be configured to obtain the quality indicators 162 from the server 160 and generate a score indicating a compliance rating for the TMF 154.

The quality indicators 162 can include a number of metrics for clinical data, including patient satisfaction scores, infection rates, readmission rates, medical error rates, and adherence to clinical guidelines rates. The system 110 can generate a score indicating a compliance rating for (i) the TMF 154, (ii) the ISF 150, (iii) the site 144, or (iv) some combination thereof. The score can be based on the instances of non-compliant data in the TMF 154 and the quality indicators 162. In this way, one instance of non-compliant data can have a greater impact on the score for compliant rating according to the quality indicators, as different types of non-compliant data can have different severity and potential impact to a clinical trial. The system 110 can be configured to generate signals based on the instances of non-compliant data in the TMF 154, each signal indicating fields and/or formats in a source document, ISF, and/or TMF that contains non-compliant data. In this way, the system 110 can be configured to identify sources of non-compliant data, e.g., sites that may not conduct phases of a clinical trial according to protocols, rules, and/or guidelines. Non-compliant data can include data that does not include all critical data fields, data in a format that is not according to the protocol for the clinical trial, may not include a signature that certifies the data in the document, among other ways to demonstrate non-compliance in a clinical trial document.

As depicted in FIG. 1B, the system 110 illustrates a TMF feedback loop 167 for updating fields or formats of the TMF 154, using one or more identified instances of non-compliant data. The NLP models 120 can be trained to identify instances of non-compliance and can generate model outputs indicating a recommendation for updating the non-compliant data to become compliant. For example, the NLP models can be configured to update clinical data in the TMF 154 by providing updated clinical data (e.g., fields, formats) to the TMF platform 154 via the TMF feedback loop 167.

FIG. 1B also depicts an example model structure for NLP models 120, which can include an input layer 164 configured to receive and process an initial set of inputs, e.g., feature data. The NLP models 120 also include an output layer 166 coupled to the input layer 164 and configured to generate a model output using output data from the input layer 164, e.g., analyzed feature data from the input layer 164. The NLP models 120 can include one or more additional layers 165 between the input layer 164 and the output layer 166, e.g., to process data between the input layer 164 and the output layer 166. The additional layers 165 can include embedding layers to generate lower-dimensional feature representations of feature data, and/or transformation layers to apply transformations to the feature data. Each of the layers can include weights, biases, and activation functions that can be updated during training of the NLP models 120. In some cases, the additional layers 165 can include normalization layers, convolution layers, recurrent layers, feedforward layers, and/or attention layers.

The system 110 described in reference to FIGS. 1A and 1B can address complexities and downstream effects of errors in source documents. These challenges include, for example, a substantial impact on the computational demands associated with evaluating of a clinical trial. For example, several computing platforms, devices, and/or computer networks can be used for the generation, monitoring, and analysis of clinical trial data to determine the results of a clinical trial. Variability, error, and other types of inaccuracy found in a source document can have a resulting impact that degrades the accuracy and reliability of clinical trial data for a clinical trial. A lack of consistency between source documents can occur for a variety of reasons, such as human error, variability on documentation practices, incomplete records, changes in medical records, different interpretations of prognosis/diagnosis based on symptoms, etc.

As an example, the inconsistency of source information between source documents can cause a degradation in data quality of clinical trial data generated from the source documents. Poor data quality can result in in downstream effects in computational processing for clinical trial platforms (including their related computing hardware and software) that process the clinical data. These effects can include issues that occur when integrating data from multiple sources and formats, extraneous consumption of resources to identify, clean, and/or pre-process inconsistent or erroneous data. The effects of errors and inconsistencies in the source documents can also propagate errors into the statistical algorithms that are applied to source data and/or any clinical trial data resulting from the source data, thereby leading to incorrect or inaccurate results. In some cases, source documents from disparate data sources of clinical trial data (such as different sites and/or phases for a clinical trial) can also present issues for analyzing clinical trial data, e.g., due to differences in formatting of source documents.

The computational complexity of reviewing and analyzing source data from source documents also exacerbates data quality and computational accuracy because the large volume of source documents generated from different contexts, e.g., phases, sites, instances, healthcare providers, of a clinical trial. Each instance of source data recording in a source document and/or generation of a source document can have a different context than another instance of source data found in another source document. Each source document includes source data for one or more entities (e.g., patients) that is recorded in a different way, e.g., at a different site, by a different provider, at a different phase of the clinical trial, than another source document that includes source data for at least one overlapping entity.

As another example, source data can also have contextual information obtained from other corpora of documents related to the clinical trial, such as trial documents and guidelines, critical data processes, ontology databases, among others. Examples of contextual data for a source document can include patient demographics, treatment information, clinical setting, timing of data collection, patient conditions, measurement methods, protocol adherence, source document origin, healthcare provider documentation and notes, and external factors, e.g., changes in healthcare provider/setting policies, equipment malfunction, and environmental factors. Thus, different source documents within a single phase, and/or across different phases, of a clinical trial can capture different contextual information (from other source documents, and/or corpora of additional documents for the clinical trial) for one or more entities.

FIG. 1C is a block diagram 170 that illustrates an example model output of the system for source document review using artificial intelligence techniques. The block diagram 170 shows an example display 104-1 that is configured by the system 110, by the NLP models 120 generating and providing the generative prompt data 140 to a client device, e.g., client device 102-1. The generative prompt data 140 configures the display 104-1 to present GUI elements and also allows for interaction with the NLP models 120.

For example, the display 104-1 includes a new session GUI element 172 (also referred to as “new session button 172”), a query history GUI element 174 (also referred to as “query history button 174”), and a number of historical query GUI elements 176-1 through 176-N (also referred to as “historical query buttons 176”). The new session button 172 allows input by the user of the client device 102 to instantiate a new session, by submitting a user input 106 to request connection to the NLP models 120 of system 110. The query history button 174 allows input by the user of the client device 102 to access previous sessions with interactions (e.g., queries, uploads of source documents) with the NLP models, by submitting a user input 106 to the system 110. The historical query buttons 176 allow for a selection of a particular session of interactions between the client device 102 and the NLP models 120. In response to the entry of a user input via a GUI element, e.g., new session button 172, query history button 174, and historical query buttons 176, the system 110 can determine a responsive output for the user input, such as providing data indicative of historical interactions with the NLP model.

The display 104-1 also shows a window 178 indicating a current session, e.g., interaction, with the NLP models 120 of the system 110. The window 178 includes a GUI element 180 that allows entry of a user input that can include a query, a mechanism for providing an electronic copy of a source document (e.g., via an attachment), and allows for entry of user feedback to any results provided via the window 178. The window 178 includes a GUI element 182 indicating a user input entered via GUI element 180, e.g., a request to identify adverse events from an input source document. In particular, the GUI element 182 includes a query “What are the adverse events in the listed document and medications used to treat them?” for input to the NLP models 120. The input query can also include a source document for input, e.g., an attachment that is provided with the input query in the GUI element 180. The window 178 shows the model output 122-1 generated in response to the query indicated by GUI element 182 (e.g., entered via GUI element 180. The model output 122-1 indicates entities and adverse events (“Congestive Heart Failure exacerbation,” and “Shortness of Breath”) along with contextual information obtained from corpora of documents other than the source document (e.g., start dates, stop dates). In some cases, the output data and its formatted can be based on contextual information obtained from corpora of documents other than the source document, e.g., using a medical ontological database to classify names of entities. A treatment such as “Medication #3” can be classified by its medication ontological classification, e.g., “Anticoagulant.”

FIG. 1D is a block diagram 190 that illustrates another example model output of the system for source document review using artificial intelligence techniques. The block diagram 190 shows an example user input of a source document, e.g., user input 106-2 or “source document input 106-2”) that can be provided to the NLP models 120 of system 110. The source document input 106-2 can include a number of fields 192-1 through 192-N (collectively “fields 192”) that indicate source data such as the clinical trial associated with the source document (e.g., “clinical trial ID”), the site for which the source data is collected (e.g., “site ID”), an identification number for the source document (e.g., “site log ID”). The source document input 106-2 can include a number of additional fields and data points for the source data.

The block diagram 190 illustrates an example model output 122-2 generated by the NLP models 120 based on the source document input 106-2. The model output 122-2 shows a chart 194 indicating events 194-1 through 194-3 (collectively “events 194”), and shows information for the events such as the status, date, and description. In this example, the source document review shown in model output 122-2 indicates three events from the source document input 106-2. The events can indicate a compliance status of data in the source document, based on analysis of the source data in the source document performed by the NLP models 120 and leveraging additional contextual information from corpora of documents related to the clinical trial. Events 194-1 and 194-3 indicate non-compliance in the source documents, such as mismatch or inconsistency for the name of a principal investigator in the source document, whereas event 194-2 indicates compliant data in the source document, e.g., a first site visit for a participant is identified as a site initiation visit.

In some cases, source document review can refer to the processing and analysis of patient data according to protocol compliance for clinical trials. For example, source data review can include improving compliance and congruence of documents according to regulatory guidelines, thereby improving patient safety during clinical trials. In some implementations, document compliance can refer to determining completeness and accuracy of content in a document and any related documents. By performing source data review, the system 110 can improve data accuracy and completeness of source data and increase the likelihood of source documents following protocols and/or other compliance rules. The system 110 can be configured mitigate risks by providing earlier detection of errors in source documents and allowing corrective actions to be performed prior to the advancement of a clinical trial from one phase to the next phase of the clinical trial. In this way, the system 110 can reduce a number of data transmissions between computing devices in a computer network for the clinical trials. The system 110 can reduce extraneous consumption of computational resources by computing devices, platforms, and networks for the clinical trial.

The system 110 can also improve inspection readiness throughout multiple instances within a phase and across phases of a clinical trial, by conducting near real-time quality review of uploaded documents as the documents are provided to the system 110. The system 110 applies natural language processing and machine learning techniques identify trends of defects, instances of events, and can provide recommendations for remedying the detected defects and/or addressing the events. In this way, the system 110 allows for proactive action to reduce non-compliant during audits of clinical trial documents and improve site compliance for clinical trial sites. The system 110 provides a computational advantage by improving timeliness in database locks between phases of a clinical trial. In some implementations, the system 110 is configured to extract entities from an input source document of a clinical trial and reconcile the entities with other instances of the entities found in other corpora of documents for the clinical trial, e.g., apply corrections to the format and/or data in the input source document. The system 110 is configured to apply a feedback loop via prompt feedback data 106-3 and the generative prompt data 140 to improve model outputs for different inputs, including instances where additional documents are provided to the system 110, e.g., in addition to updated prompts from client devices 102. In some cases, the system 110 can be configured to continually update the generative prompt data 140 in response to receiving user inputs and/or additional documents, e.g., a source document, a corpora of documents.

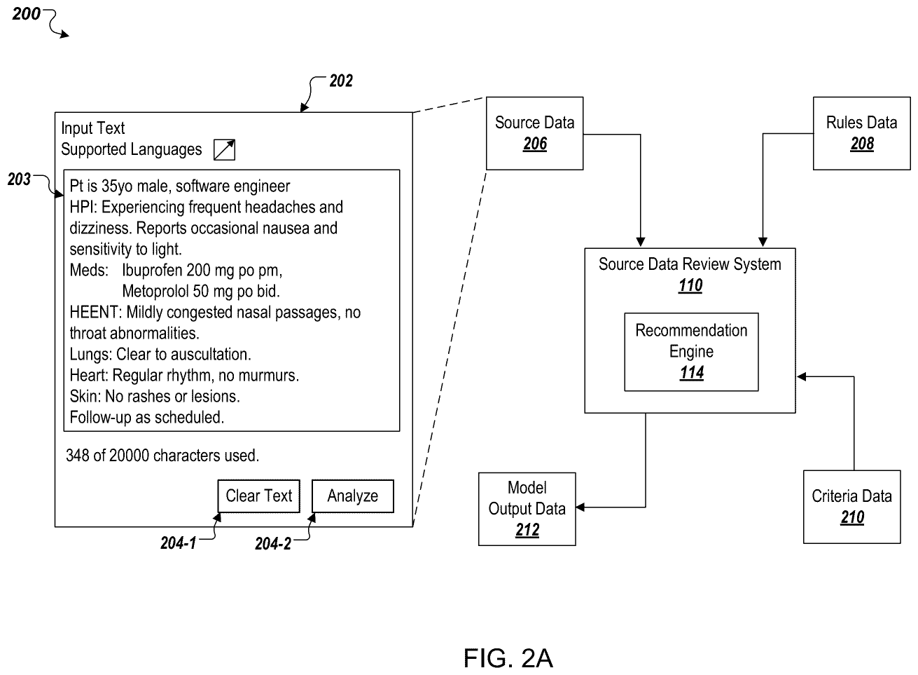

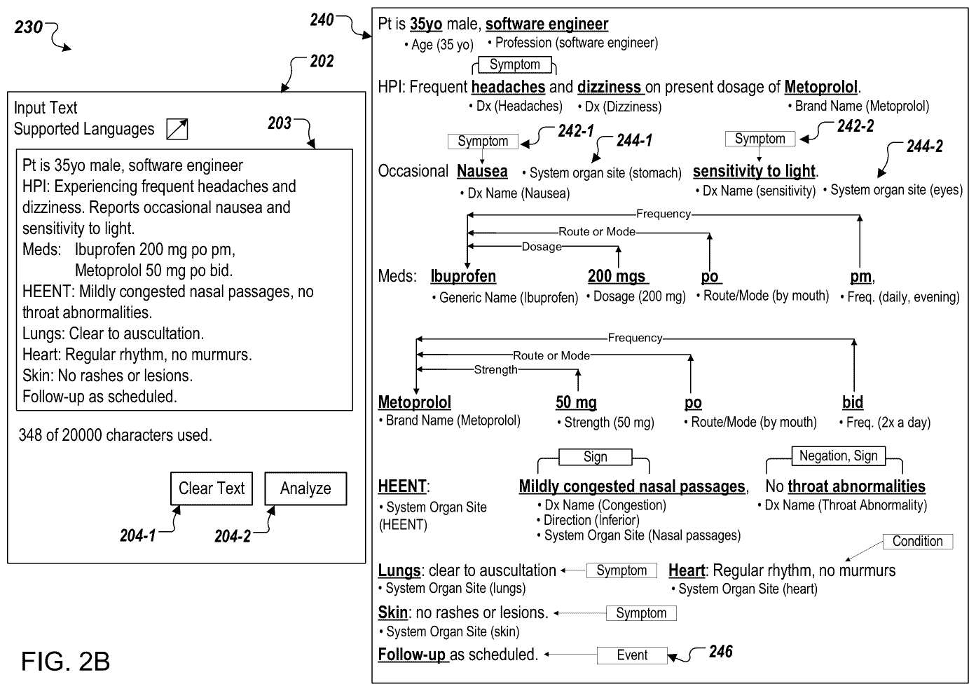

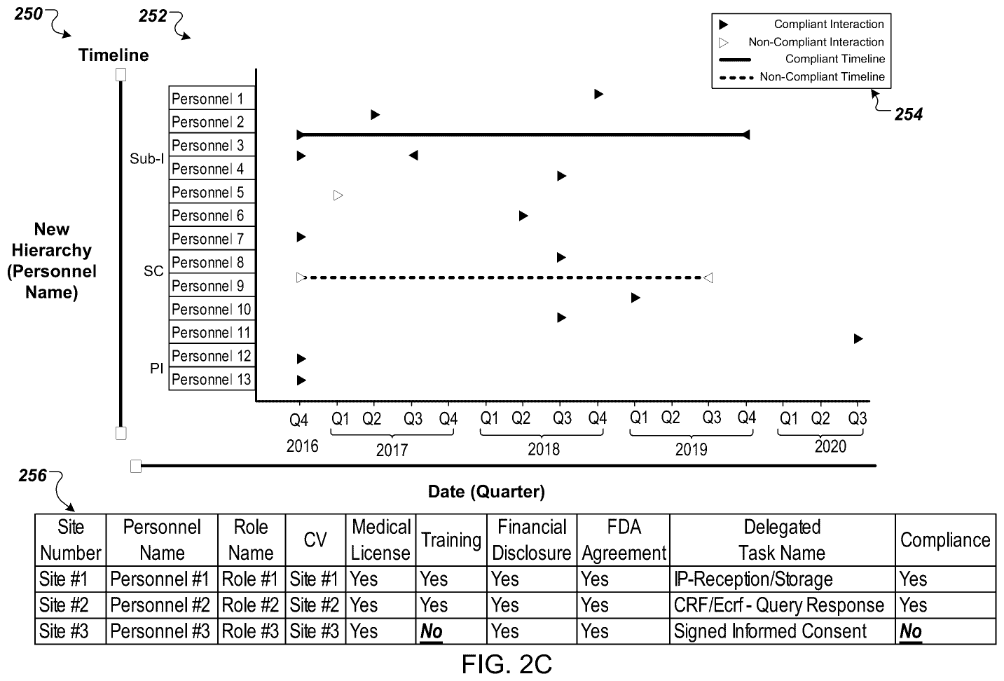

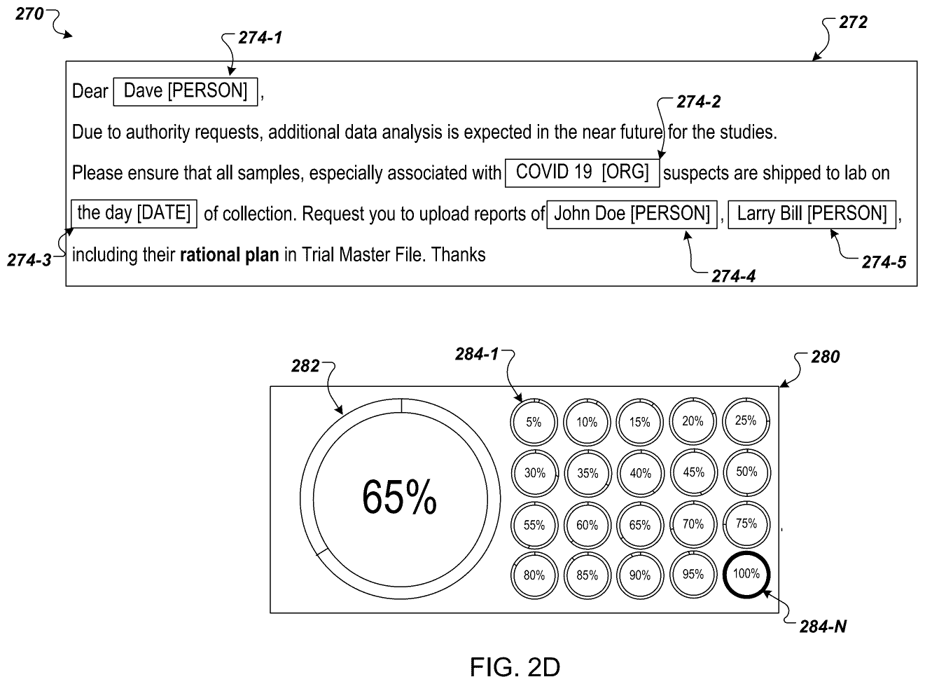

FIG. 2A is a diagram that illustrates an example input for the source document review system of FIGS. 1A and 1B. The diagram 200 shows a GUI 202 that can be presented on a display, e.g., display 104, of a client device, e.g., client device 102. The GUI 202 shows input text 203 that can be found in a source document for a clinical trial, and the input text 203 can also be referred to as a user input 106. The input text 203 shows the information that may be collected and stored in the source document, e.g., by a clinician for a participant of the clinical trial. The GUI 202 also includes GUI elements 204-1 and 204-2, shown as buttons that the user to “clear text” and “analyze” (respectively) the input text shown in the GUI 203. The GUI element 204-1 can be referred to as the “clear text” button, while the GUI element 204-2 can be referred to as the “analyze button.” The control button 204-1 presented by GUI element 204-1 can provide a selectable control that clears the input text 203 shown in the GUI 202. The analyze button presented by GUI element 204-2 can provide a selectable control that transmits the input text 203 to the system 110. The diagram 200 shows the input text 203 as an example of source data 206 that can be provided for input to the engine 114 of the system 110.

In addition to the source data 206, the system 110 can also obtain rules data 208 and criteria data 210 to generate model output data 212. The model output data 212 depicted in FIG. 2A can be an example of model output 122 described in reference to FIGS. 1A and 1B above. The rules data 208 can be an example of guidelines and/or regulations for conducting the clinical trial according to ethical and/or legal standards. The criteria data 210 can be an example of conditions for the clinical trial to determine eligibility in the clinical trial, as well as outcomes. Examples of criteria can include parameters for excluding participants, such as a medication, medical history, demographics, or some combination thereof, that render the participant ineligible for participating in the clinical trial. The criteria data 210 can also include particular outcome metrics for measuring the efficacy and/or efficiency of the clinical trial. The system 110, through the NLP models 120 (shown in FIGS. 1A and 1B above), performs an evaluation of site documents to evaluate adherence to of the clinical trial meets to the rules data 208 and the criteria data 210.

FIG. 2B is a diagram that illustrates an example output of the system of FIGS. 1A and 1B. The diagram 230 shows an example GUI 202 of FIG. 2A with input text 203 and GUI elements 204-1 and 204-2. As described in reference to FIG. 2A above, the input text 203 can be referred to as a user input, e.g., user input 106 of FIG. 1A. The system 110 can be configured to generate a GUI 240 as an output, with annotations of the text found in the input text 203 indicating different entities and respective classes according to a medical ontology. Examples of entities can include symptoms, signs (positive or negative indicators), conditions, etc. As shown by GUI element 242-1 and 242-2 in the GUI 240, the output of the system 110 shows a classification of symptoms, e.g., “nausea,” and “sensitivity to light,” respectively, while GUI elements 244-1 and 244-2 indicate organ sites (i.e., where on the patient's body the symptom appears or presents itself) for the patient, e.g., “stomach” and “eyes”, respectively. Other examples of annotations representing entities in the GUI 240 can include strength of medication, dosage of medication, route or mode for taking medication, frequency and/or time of day, type of brand for the medication, among other classifications for entities.

The GUI 240 also shows a GUI element 246 indicating an event among the entities (e.g., symptoms, signs, etc.) identified in the input text 203. The GUI element 246 corresponds to a detected event indicating a follow-up appointment to adhere to clinical trial protocols that is missing from the clinical trial. In this way, the detected event can be transmitted to a client device and allow for corrective action to be taken so that the clinical trial can adhere to clinical trial protocols. The detected event can be generated, at least in part, on data from different corpora of documents, such as other types of records in databases.

FIG. 2C is a diagram that illustrates an example output of the system of FIGS. 1A and 1B. The diagram 250 shows an example chart 252 for determining compliance of clinical trial sites, with legend 254 indicating compliance status of different clinician (depicted as personnel in FIG. 2C) through phases of a clinical trial. The chart 252 shows compliant interactions for the clinicians at a time instance of the clinical trial depicted in FIG. 2C as solid black triangles. Non-compliant interactions (e.g., events) detected by the system 110 are shown in FIG. 2C as solid white triangles in the chart 252. The chart 252 also shows a timeline throughout multiple time instances of interactions, e.g., duration of involvement for a clinician in the clinical trial. as solid black lines for compliant clinicians and dashed lines for non-compliant clinicians. In some cases, compliance for a clinician can indicate that documents for a clinician participating in the clinical trial are valid, e.g., the data in the document is compliant. In some cases, non-compliance for a clinician can indicate that documents for the clinician are not electronically stored. In these cases, the document can be a handwritten document that is yet to be digitized by a timeframe for a phase of the clinical trial.

For example, the diagram 250 also shows an example table 256 indicating different site numbers and personnel, along with other metadata base for the personnel. The system 110 can be configured to determine compliance on a role-by-role and site-by-site basis. The table 256 shows that for site #3, the clinician (e.g., “personnel #3”) did not complete training and thus indicates an instance of non-compliance in the clinical trial. In some cases, the non-compliance can be a result of a lack of digitization for a particular document type. The table 256 can also provide a summary of compliance status for different document types, such as medical licenses, agreements, training documents, resume/curriculum vitae documents, referrals, reference documents, etc. Referring to FIGS. 1A and 1B, the system 110 can be configured to provide a table, e.g., table 256, for output by the client device 102-1 to provide an overview of compliance across different document types.

The system 110 can be configured extract data from documents, e.g., handwritten and/or typed documents, and reconcile the extracted data with corpora of documents from different databases, clinical trial systems and computing devices, e.g., TMF platforms and IMF platforms. For example, the techniques can provide that all clinical study personnel names listed in table 256 are documented in a delegation log, e.g., a log that indicates tasks that are to be performed by each clinician. The disclosed techniques can also provide an indication from numerous site documents to confirm that each clinician has the completed training to perform their delegated tasks, and that the clinician submitted documents to perform the clinical trial according to the guidelines and rules for the clinical trial. These documents can include disclosures, certifications, educational history, licensing, certifications, etc.

The NLP models 120 of the system 110 can be configured to apply natural language processing to evaluate and apply compliance rules to data in documents to identify sources of non-compliance, are identified in the data from the documents. In some implementations, the NLP models 120 can be configured to apply named entity recognition algorithm to the data from site documents to identify and modify data in the documents, such as redacting, formatting, etc.

FIG. 2D is a diagram that illustrates another example output of the system of FIGS. 1A and 1B. The diagram 270 shows an example GUI 272 that includes a number of GUI elements 274-1 through 274-5 (collectively referred to as “GUI elements 274”). The GUI elements 274 illustrate different examples of entities found in source documents, generated with contextual information from different corpora of documents.

The system 110 can also generate an output 280 showing GUI element 282 and GUI elements 284-1 to 284-N (collectively “GUI elements 284”). The GUI element 282 indicates an average compliance score (e.g., “65%”) for the clinical trial across all clinical trial sites. The GUI elements 284 show compliance scores for each clinical trial site. For example, a first clinical trial site for the clinical trial is shown with a score of “5%” indicated by GUI element 284-1. As another example, the last clinical trial site for the clinical trial is shown with a score of “100%” indicated by GUI element 284-N.

The output 280 provides a comparison of detect rates across all clinical sites to identify high-risk sites, indicating clinical sites that are more likely to have documents with non-compliant data, e.g., compared to other clinical sites. In this way, the system 110 can allow for mitigating risks found in auditing and inspection processes for site documents. Thus, the system allows for proactive monitoring of the clinical sites, to improve compliance by preventing instances of non-compliance while studies and clinical trials are performed at the site, e.g., in contrast to conducting site visits during checkpoints of the clinical trial corresponding to the end of one phase and the beginning of the next phase. The preventative actions provided by the system 110 can improve rates and timeliness of database lock for documents in clinical trials. A phase of a clinical trial may not proceed without a database lock of documents from a previous phase of the clinical trial, e.g., propagating delays in the clinical trial.

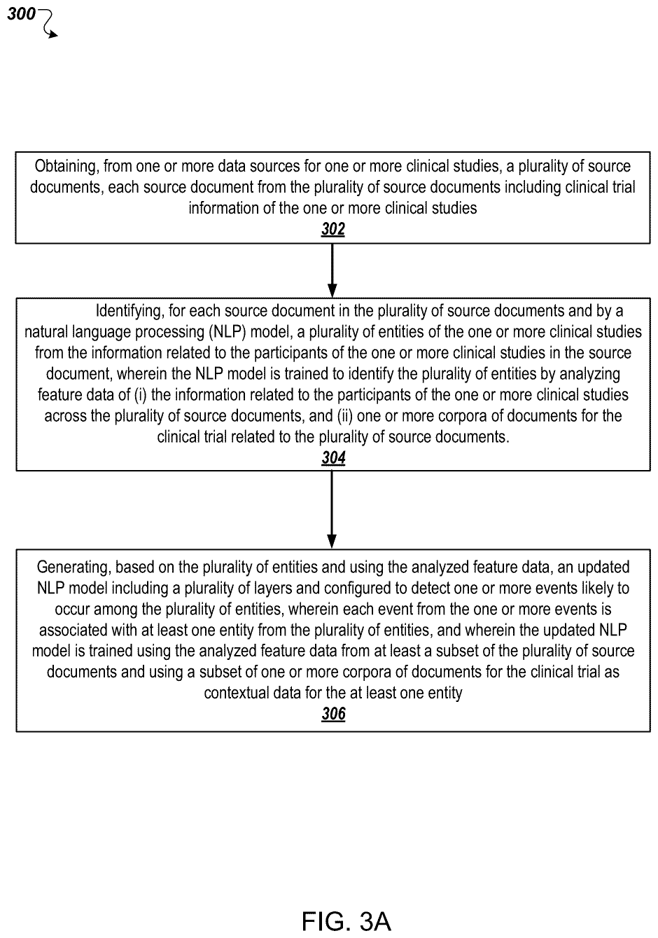

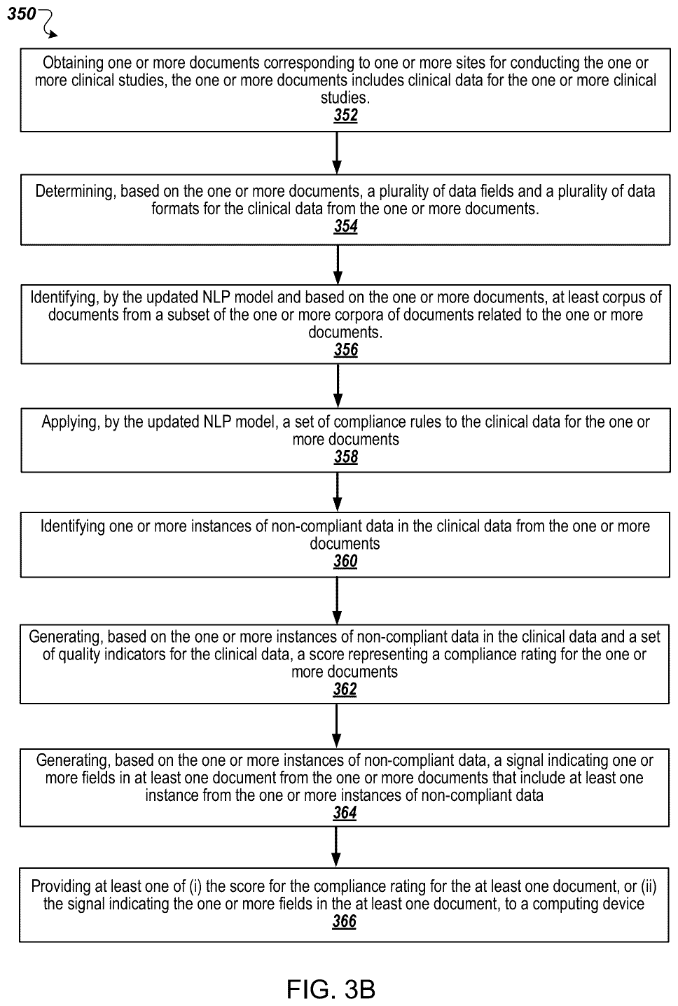

FIG. 3A is a flow diagram that illustrates an example of a process 300 for detecting events that have occurred between entities of source documents for a clinical study. The process 300 can be performed by computing devices and/or systems, such as the source data review system 110 and computing device 105 illustrated in FIGS. 1A and 1B. Examples of

The process 300 includes obtaining, from one or more data sources for one or more clinical studies, a plurality of source documents, each source document from the plurality of source documents including clinical trial information of the one or more clinical studies (302). Examples of source documents can include data obtained from databases 112 described in reference to FIG. 1A above but can also include a source document 106-2 provided for input, e.g., user inputs 106.

The process 300 includes identifying, for each source document in the plurality of source documents and by an NLP model, a plurality of entities of the one or more clinical studies from the information related to the participants of the one or more clinical studies in the source document (304). The NLP model is trained to identify the plurality of entities by analyzing feature data of (i) the information related to the participants of the one or more clinical studies across the plurality of source documents, and (ii) one or more corpora of documents for the clinical trial related to the plurality of source documents. Examples of the NLP models can include NLP models 120 of the recommendation engine 114 described in reference to FIG. 1A above. The NLP models can be trained by a model training module 124 to analyze feature data from corpora of documents, such as documents obtained from the databases 112.

The process 300 includes generating, based on the plurality of entities and using the analyzed feature data, an updated NLP model including a plurality of layers and configured to detect one or more events likely to have occurred among the plurality of entities (306). Each event from the one or more events is associated with at least one entity from the plurality of entities and the updated NLP model is trained using the analyzed feature data from at least a subset of the plurality of source documents and using a subset of one or more corpora of documents for the clinical trial as contextual data for the at least one entity. The recommendation engine 114 of FIG. 1A can be configured to generate an updated NLP model 132 as a model different than the NLP models 120 or can be configured to update the parameters of NLP models 120 to generate the updated NLP model 132. The updated NLP model can be configured to update one or more parameters of at least one layer from the plurality of layers in response to receiving a user input representing feedback to a model output and/or generative prompt data from the updated NLP model. Examples of model outputs can include a signal generated by an NLP model, the signal indicating the detection of an occurrence of one or more events related to entities in a user input, e.g., a query 106-1.

In some implementations, generating the updated NLP model includes generating, for each source document in the plurality of source documents, a classification of each respective entity from the plurality of entities. The classification can indicate a class of medical ontology for the respective entity based on the analyzed feature data.

In some implementations, the process 300 includes receiving, from a computing device (e.g., a client device 102-1) communicatively coupled to the one or more computers, an input query, e.g., query 106-1, related to at least one entity from the plurality of entities. The process 300 can include generating, based on the input query and by the updated NLP model, a signal indicating one or more events associated with the at least one entity.

In some implementations, one or more events from the events detected by the updated NLP model can indicate a correlation between two or more entities from the plurality of entities. A correlation can indicate a likelihood of association between the two or more entities, in which a strong correlation (e.g. a likelihood value close to 1) indicates a higher degree of association than a weak correlation (e.g., a likelihood value close to zero) indicating a weak degree of association.

In some implementations, the clinical trial information includes at least one of (i) data related to participants, (ii) data related to clinicians, (iii) data related to study protocols, or (iv) data related to regulations, for the one or more clinical studies.

In some implementations, the updated NLP model is configured to generate a plurality of events likely to have occurred among the plurality of entities. The updated NLP model can be configured to generate, for each event in the plurality of events, a value indicating a likelihood of association of entities from at least a subset of the plurality of entities.

In some implementations, training the updated NLP model includes providing a training example query for input to the updated NLP model and generating, using the training example query and by the updated NLP model, a training model output representing one or more detected events associated with plurality of entities. Training the updated NLP model can include obtaining ground truth data indicating one or more events associated with the plurality of entities, determining a score based on a comparison of the ground truth data and the training model output, and based on the score exceeding a threshold, updating one or more parameters of at least one layer from the plurality of layers.

In some implementations, the process 300 includes detecting, using the updated NLP engine, an adverse event from the plurality of events. The adverse event can indicate that the information related to the participants does not follow a protocol from the one or more protocols for conducting the one or more clinical studies. The process 300 can include, in response to detecting the adverse event, generating data indicative of one or more updates to the information related to the participants found in the source document from the plurality of source documents that includes an entity associated with the adverse event.

In some implementations, the process 300 includes, generating, by the updated NLP model, generative prompt data that configures a user interface of a client device. The generative prompt data cause display of a visual representation of annotations corresponding to each respective entity from the plurality of entities, e.g., generative prompt data 140. Each annotation indicates the class of medical ontology for the respective entity and the NLP model is trained to generate the generative prompt data using one or more generative visualization techniques.

In some implementations, the process 300 includes, providing the generative prompt data to the client device, e.g., client device 102. By providing the generative prompt data to the client device, the generative prompt data causes the client device to update the user interface to include one or more graphical elements, each graphical element corresponding to each annotation from the annotations.

In some implementations, the process 300 includes providing, for output by the one or more computers, the user interface including a respective selectable control for providing feedback to an identification of an event from the one or more events for the one or more clinical studies, the identified event corresponding to a graphical element from the one or more graphical elements. The respective selectable control can be a feedback mechanism to provide feedback to the recommendation engine 114 to train and/or update the NLP models 120 or 132. The process 300 can include receiving, by the user interface, a user selection of one or more of the selectable controls included in the user interface and updating one or more parameters of the plurality of layers for the updated NLP model. Examples of the updated parameters can include model parameters 128.

In some implementations, the process 300 includes determining, from the plurality of events and using the NLP model, one or more instances of non-compliant data in at least one source document from the plurality of source documents. The non-compliant data is associated with an entity from the plurality of entities. An instance from the one or more instances of non-compliance can include a deviation from at least one protocol from one or more protocols for conducting the one or more clinical studies. An instance from the one or more instances of non-compliant data can also indicate a treatment plan that does not follow protocol for the one or more clinical studies.

In some implementations, the process 300 includes identifying, based one or more instances of non-compliant data, an output trend indicating a pattern of non-compliance for the one or more clinical studies. The pattern can be associated with at least one (i) a subset of entities from the plurality of entities, or (ii) one or more sites for conducting the one or more clinical studies.

In some implementations, the process 300 includes monitoring, by the updated NLP models, input data from a computing device communicatively coupled to the one or more computer systems. The process 300 can include detecting, from the input data and by the updated NLP models, a detection of non-compliant data in the input data. The process 300 can include generating, based on the detection of the non-compliant data and using the updated NLP models, one or more of (i) a signal indicating the detection of the non-compliant data, or (ii) at least one adjustment for the non-compliant data.

FIG. 3B is a flow diagram that illustrates an example of a process 350 for generating signals indicating non-compliant data in source documents. The process 350 can be performed by computing devices and/or systems, such as the source data review system 110 and computing device 105 illustrated in FIGS. 1A and 1B.

The process 350 includes obtaining one or more documents corresponding to one or more sites for conducting the one or more clinical studies, the one or more documents including clinical data for the one or more clinical studies (352). The one or more documents can include at least one of (i) certification records, (ii) delegation tasks, (iii) training logs, (iv) financial disclosures, or (v) a set of protocols.

The process 350 includes determining, based on the one or more documents, a plurality of data fields and a plurality of data formats for the clinical data from the one or more documents (354).

The process 350 includes identifying, by the updated NLP model and based on the one or more documents, at least corpus of documents from a subset of the one or more corpora of documents related to the one or more documents (356).

The process 350 includes applying, by the updated NLP model, a set of compliance rules to the clinical data for the one or more documents (358).

The process 350 includes identifying one or more instances of non-compliant data in the clinical data from the one or more documents (360).

The process 350 includes generating, based on the one or more instances of non-compliant data in the clinical data and a set of quality indicators for the clinical data, a score representing a compliance rating for the one or more documents (362).

The process 350 includes generating, based on the one or more instances of non-compliant data, a signal indicating one or more fields in at least one document from the one or more documents that include at least one instance from the one or more instances of non-compliant data (364). In some implementations, the updated NLP model is configured to identify a trend from the one or more instances of non-compliant data in the clinical data. The trend can indicate that a document from the one or more documents that do not meet at least one protocol from the one or more protocols or at least one rule in the set of compliance rules.

The process 350 includes providing at least one of (i) the score for the compliance rating for the at least one document, or (ii) the signal indicating the one or more fields in the at least one document, to a computing device (366).

In some implementations, the process 350 can include determining, by the updated NLP model and based on the set of compliance rules, a non-compliance rate of a set of documents, the set of documents associated with a site from the one or more sites and a threshold value for non-compliant data for the site. The process 350 can include comparing the non-compliance rate to a threshold value for non-compliant data for the site and based on the non-compliance rate to the threshold value, providing the signal indicating the one or more instances of non-compliant data in the set of documents to a computing device.

In some implementations, the process 350 can include analyzing, by the updated NLP model, the one or more documents. The analysis of the source documents can include comparing one or more site fields in the one or more documents to one or more fields in the one or more corpora of documents. The process 350 can include based on the comparison, generating a set of indicators for a set of fields, each indicator in the set of indicators corresponding to a field in the set of fields. The indicator from the set of indicators for the field in the set of fields represents compliance status of data represented by the field.

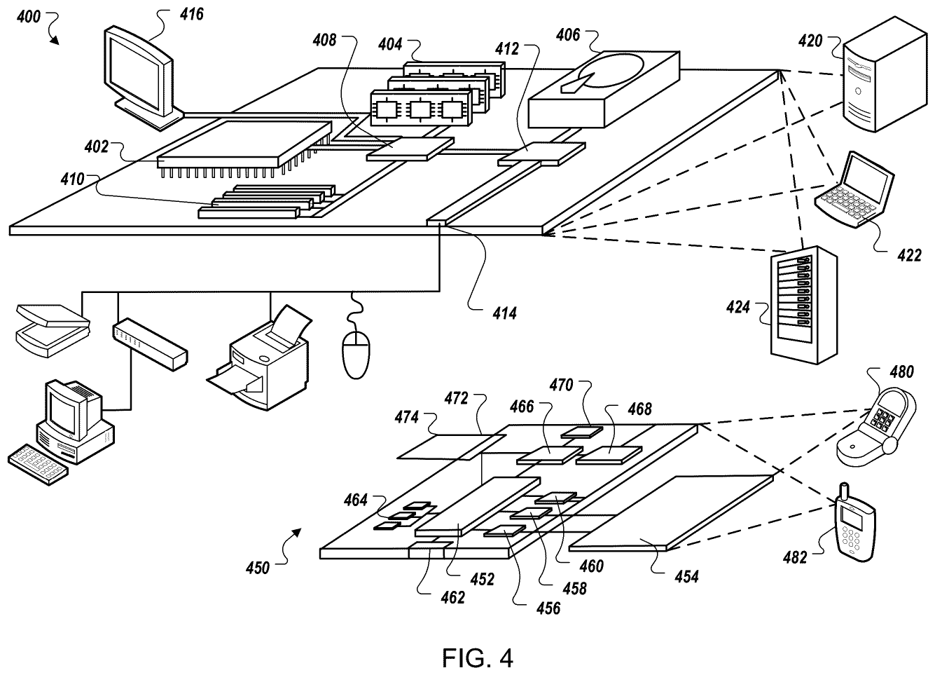

FIG. 4 is a block diagram of computing devices 400, 450 that may be used to implement the systems and methods described in this document, as either a client or as a server or multiple servers. As an example, computing device 105, source data review system 110, client devices 102, and/or server 160 can be an example of computing devices 400, 450 to analyze source documents and detect occurrences of events associated with entities of the source documents of the clinical studies. Computing device 400 and 450 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations described and/or claimed in this document.

Computing device 400 includes a processor 402, memory 404, a storage device 406, a high-speed interface 408 connecting to memory 404 and high-speed expansion ports 410, and a low speed interface 412 connecting to low speed bus 414 and storage device 406. Each of the components 402, 404, 406, 408, 410, and 412, are interconnected using various busses, and may be mounted on a common motherboard or in other manners as appropriate. The processor 402 can process instructions for execution within the computing device 400, including instructions stored in the memory 404 or on the storage device 406 to display graphical information for a GUI on an external input/output device, such as display 416 coupled to high speed interface 408. In other implementations, multiple processors and/or multiple buses may be used, as appropriate, along with multiple memories and types of memory. Also, multiple computing devices 400 may be connected, with each device providing portions of the necessary operations, e.g., as a server bank, a group of blade servers, or a multi-processor system.

The memory 404 stores information within the computing device 400. In one implementation, the memory 404 is a computer-readable medium. In one implementation, the memory 404 is a volatile memory unit or units. In another implementation, the memory 404 is a non-volatile memory unit or units.

The storage device 406 is capable of providing mass storage for the computing device 400. In one implementation, the storage device 406 is a computer-readable medium. In various different implementations, the storage device 406 may be a floppy disk device, a hard disk device, an optical disk device, or a tape device, a flash memory or other similar solid-state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer- or machine-readable medium, such as the memory 404, the storage device 406, or memory on processor 402.

The high-speed controller 408 manages bandwidth-intensive operations for the computing device 400, while the low speed controller 412 manages lower bandwidth-intensive operations. Such allocation of duties is exemplary only. In one implementation, the high-speed controller 408 is coupled to memory 404, display 416, e.g., through a graphics processor or accelerator, and to high-speed expansion ports 410, which may accept various expansion cards (not shown). In the implementation, low-speed controller 412 is coupled to storage device 406 and low-speed expansion port 414. The low-speed expansion port, which may include various communication ports, e.g., USB, Bluetooth, Ethernet, wireless Ethernet, may be coupled to one or more input/output devices, such as a keyboard, a pointing device, a scanner, or a networking device such as a switch or router, e.g., through a network adapter.

The computing device 400 may be implemented in a number of different forms, as shown in the figure. For example, it may be implemented as a standard server 420, or multiple times in a group of such servers. It may also be implemented as part of a rack server system 424. In addition, it may be implemented in a personal computer such as a laptop computer 422. Alternatively, components from computing device 400 may be combined with other components in a mobile device (not shown), such as device 450. Each of such devices may contain one or more of computing device 400, 450, and an entire system may be made up of multiple computing devices 400, 450 communicating with each other.

Computing device 450 includes a processor 452, memory 464, an input/output device such as a display 454, a communication interface 466, and a transceiver 468, among other components. The device 450 may also be provided with a storage device, such as a microdrive or other device, to provide additional storage. Each of the components 450, 452, 464, 454, 466, and 468, are interconnected using various buses, and several of the components may be mounted on a common motherboard or in other manners as appropriate.

The processor 452 can process instructions for execution within the computing device 450, including instructions stored in the memory 464. The processor may also include separate analog and digital processors. The processor may provide, for example, for coordination of the other components of the device 450, such as control of user interfaces, applications run by device 450, and wireless communication by device 450.

Processor 452 may communicate with a user through control interface 458 and display interface 456 coupled to a display 454. The display 454 may be, for example, a TFT LCD display or an OLED display, or other appropriate display technology. The display interface 456 may include appropriate circuitry for driving the display 454 to present graphical and other information to a user. The control interface 458 may receive commands from a user and convert them for submission to the processor 452. In addition, an external interface 462 may be provided in communication with processor 452, so as to enable near area communication of device 450 with other devices. External interface 462 may provide, for example, for wired communication, e.g., via a docking procedure, or for wireless communication, e.g., via Bluetooth or other such technologies.

The memory 464 stores information within the computing device 450. In one implementation, the memory 464 is a computer-readable medium. In one implementation, the memory 464 is a volatile memory unit or units. In another implementation, the memory 464 is a non-volatile memory unit or units. Expansion memory 474 may also be provided and connected to device 450 through expansion interface 472, which may include, for example, a SIMM card interface. Such expansion memory 474 may provide extra storage space for device 450, or may also store applications or other information for device 450. Specifically, expansion memory 474 may include instructions to carry out or supplement the processes described above, and may include secure information also. Thus, for example, expansion memory 474 may be provided as a security module for device 450, and may be programmed with instructions that permit secure use of device 450. In addition, secure applications may be provided via the SIMM cards, along with additional information, such as placing identifying information on the SIMM card in a non-hackable manner.

The memory may include for example, flash memory and/or MRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 464, expansion memory 474, or memory on processor 452.

Device 450 may communicate wirelessly through communication interface 466, which may include digital signal processing circuitry where necessary. Communication interface 466 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 468. In addition, short-range communication may occur, such as using a Bluetooth, WiFi, or other such transceiver (not shown). In addition, GPS receiver module 470 may provide additional wireless data to device 450, which may be used as appropriate by applications running on device 450.

Device 450 may also communicate audibly using audio codec 460, which may receive spoken information from a user and convert it to usable digital information. Audio codec 460 may likewise generate audible sound for a user, such as through a speaker, e.g., in a handset of device 450. Such sound may include sound from voice telephone calls, may include recorded sound, e.g., voice messages, music files, etc., and may also include sound generated by applications operating on device 450.

The computing device 450 may be implemented in a number of different forms, as shown in the figure. For example, it may be implemented as a cellular telephone 480. It may also be implemented as part of a smartphone 482, personal digital assistant, or other similar mobile device.

Various implementations of the systems and techniques described here can be realized in digital electronic circuitry, integrated circuitry, specially designed ASICs, computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs, also known as programs, software, software applications or code, include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back-end component, e.g., as a data server, or that includes a middleware component such as an application server, or that includes a front-end component such as a client computer having a GUI or a Web browser through which a user can interact with an implementation of the systems and techniques described here, or any combination of such back-end, middleware, or front-end components. The components of the system can be interconnected by any form or medium of digital data communication such as, a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

In addition, certain data may be treated in one or more ways before it is stored or used, so that personally identifiable information is removed. For example, in some embodiments, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

A number of embodiments have been described. Nevertheless, it will be understood that various modifications may be made without departing from the spirit and scope of the invention. Accordingly, other embodiments are within the scope of the following claims. While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment.

Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, some processes depicted in the accompanying figures do not necessarily require the particular order shown, or sequential order, to achieve desirable results.

What is claimed is:

1. A computer-implemented method performed by one or more computers, the computer-implemented method comprising:
obtaining, from one or more data sources for one or more clinical studies, a plurality of source documents, wherein each source document from the plurality of source documents comprises clinical trial information of the one or more clinical studies;
identifying, for each source document in the plurality of source documents and by a natural language processing (NLP) model, a plurality of entities of the one or more clinical studies from the information related to participants of the one or more clinical studies in the source document, wherein the NLP model is trained to identify the plurality of entities by analyzing feature data of (i) the information related to the participants of the one or more clinical studies across the plurality of source documents, and (ii) one or more corpora of documents for the clinical trial related to the plurality of source documents; and
generating, based on the plurality of entities and using the analyzed feature data, an updated NLP model comprising a plurality of layers and configured to detect one or more events likely to have occurred among the plurality of entities, wherein each event from the one or more events is associated with at least one entity from the plurality of entities, and wherein the updated NLP model is trained using the analyzed feature data from at least a subset of the plurality of source documents and using a subset of one or more corpora of documents for the clinical trial as contextual data for the at least one entity,
wherein the updated NLP model is configured to update one or more parameters of at least one layer from the plurality of layers in response to receiving a user input representing feedback to a model output from the updated NLP model.

2. The computer-implemented method of claim 1, further comprising:
receiving, from a computing device communicatively coupled to the one or more computers, an input query related to at least one entity from the plurality of entities;
generating, based on the input query and by the updated NLP model, a signal representing one or more events associated with the at least one entity.

3. The computer-implemented method of claim 1, wherein at least one event from the one or more events detected by the updated NLP model indicates a correlation between two or more entities from the plurality of entities.

4. The computer-implemented method of claim 1, wherein the clinical trial information comprises at least one of (i) data related to participants, (ii) data related to clinicians, (iii) data related to study protocols, or (iv) data related to regulations, for the one or more clinical studies.

5. The computer-implemented method of claim 1, wherein generating the updated NLP model comprises generating, for each source document in the plurality of source documents, a classification of each respective entity from the plurality of entities, wherein the classification indicates a class of medical ontology for the respective entity based on the analyzed feature data.

6. The computer-implemented method of claim 1, wherein the updated NLP model is configured to generate a plurality of events likely to have occurred among the plurality of entities, wherein the updated NLP model is configured to generate, for each event in the plurality of events, a value indicating a likelihood of association of entities from at least a subset of the plurality of entities.

7. The computer-implemented method of claim 1, wherein training the updated NLP model comprises:
providing a training example query for input to the updated NLP model;
generating, using the training example query and by the updated NLP model, a training model output representing one or more detected events associated with plurality of entities;
obtaining ground truth data, wherein the ground truth data indicates one or more events associated with the plurality of entities;
determining a score based on a comparison of the ground truth data and the training model output; and
based on the score exceeding a threshold, updating one or more parameters of at least one layer from the plurality of layers.

8. The computer-implemented method of claim 1, further comprising:
detecting, using the updated NLP model, an adverse event from the one or more events, wherein the adverse event indicates that the information related to the participants does not follow a protocol from the one or more protocols for conducting the one or more clinical studies; and
in response to detecting the adverse event, generating data indicative of one or more updates to the information related to the participants found in the source document from the plurality of source documents that includes an entity associated with the adverse event.

9. The computer-implemented method of claim 1, comprising:
generating, by the updated NLP model, generative prompt data that configures a user interface of a client device, wherein the generative prompt data causes display of a visual representation of annotations corresponding to each respective entity from the plurality of entities, wherein each annotation indicates a class of medical ontology for the respective entity, and wherein the NLP model is trained to generate the generative prompt data using one or more generative visualization techniques.

10. The computer-implemented method of claim 9, comprising:
providing the generative prompt data to the client device, wherein providing the generative prompt data causes the client device to update the user interface to include one or more graphical elements, each graphical element corresponding to each annotation from the annotations.

11. The computer-implemented method of claim 10, comprising:
providing, for output by the one or more computers, the user interface including a respective selectable control for providing feedback to an identification of an event from the one or more events for the one or more clinical studies, the identified event corresponding to a graphical element from the one or more graphical elements;
receiving, by the user interface, a user selection of one or more of the selectable controls included in the user interface; and
updating one or more parameters of the plurality of layers for the updated NLP model.

12. The computer-implemented method of claim 1, comprising:
determining, from the one or more events and using the NLP model, one or more instances of non-compliant data in at least one source document from the plurality of source documents, wherein the non-compliant data is associated with an entity from the plurality of entities.

13. The computer-implemented method of claim 12, wherein an instance from the one or more instances of non-compliance comprises a deviation from at least one protocol from one or more protocols for conducting the one or more clinical studies.

14. The computer-implemented method of claim 12, wherein an instance from the one or more instances of non-compliant data indicates a treatment plan that does not follow protocol for the one or more clinical studies.

15. The computer-implemented method of claim 1, comprising:
identifying, based one or more instances of non-compliant data, an output trend indicating a pattern of non-compliance for the one or more clinical studies, the pattern being associated with at least one (i) a subset of entities from the plurality of entities, or (ii) one or more sites for conducting the one or more clinical studies.

16. The computer-implemented method of claim 1 comprising:
obtaining one or more documents corresponding to one or more sites for conducting the one or more clinical studies, the one or more documents comprising clinical data for the one or more clinical studies;
determining, based on the one or more documents, a plurality of data fields and a plurality of data formats for the clinical data from the one or more documents;
identifying, by the updated NLP model and based on the one or more documents, at least corpus of documents from a subset of the one or more corpora of documents related to the one or more documents;
applying, by the updated NLP model, a set of compliance rules to the clinical data for the one or more documents, wherein applying the set of compliance rules comprises:
identifying one or more instances of non-compliant data in the clinical data from the one or more documents;
generating, based on the one or more instances of non-compliant data in the clinical data and a set of quality indicators for the clinical data, a score representing a compliance rating for the one or more documents; and
generating, based on the one or more instances of non-compliant data, a signal indicating one or more fields in at least one document from the one or more documents that include at least one instance from the one or more instances of non-compliant data; and
providing at least one of (i) the score for the compliance rating for the at least one document, or (ii) the signal indicating the one or more fields in the at least one document, to a computing device.

17. The computer-implemented method of claim 16, wherein the one or more documents comprises at least one of (i) certification records, (ii) delegation tasks, (iii) training logs, (iv) financial disclosures, or (v) a set of protocols.

18. The computer-implemented method of claim 16, wherein the updated NLP model is configured to identify a trend from the one or more instances of non-compliant data in the clinical data, the trend indicating the one or more documents that do not meet at least one protocol from the one or more protocols or at least one rule in the set of compliance rules.

19. The computer-implemented method of claim 16, comprising:
determining, by the updated NLP model and based on the set of compliance rules, a non-compliance rate of a set of documents, the set of documents associated with a site from the one or more sites and a threshold value for non-compliant data for the site;
comparing the non-compliance rate to a threshold value for non-compliant data for the site; and
based on the non-compliance rate to the threshold value, providing the signal indicating the one or more instances of non-compliant data in the set of documents to a computing device.

20. The computer-implemented method of claim 16, comprising:
analyzing, by the updated NLP model, the one or more documents, wherein analyzing the one or more documents includes comparing one or more site fields in the one or more documents to one or more fields in the one or more corpora of documents; and
based on the analyzing of the one or more documents, generating a set of indicators for a set of fields, each indicator in the set of indicators corresponding to a field in the set of fields, wherein the indicator from the set of indicators for the field in the set of fields represents compliance status of data represented by the field.

21. A source document review system comprising:
a computing device comprising at least one processor; and
a memory communicatively coupled to the at least one processor, the memory storing instructions which, when executed by the at least one processor, cause the at least one processor to perform operations comprising:
obtaining, from one or more data sources for one or more clinical studies, a plurality of source documents, wherein each source document from the plurality of source documents comprises clinical trial information of the one or more clinical studies;
identifying, for each source document in the plurality of source documents and by a natural language processing (NLP) model, a plurality of entities of the one or more clinical studies from the information related to participants of the one or more clinical studies in the source document, wherein the NLP model is trained to identify the plurality of entities by analyzing feature data of (i) the information related to the participants of the one or more clinical studies across the plurality of source documents, and (ii) one or more corpora of documents for the clinical trial related to the plurality of source documents; and
generating, based on the plurality of entities and using the analyzed feature data, an updated NLP model comprising a plurality of layers and configured to detect one or more events likely to have occurred among the plurality of entities, wherein each event from the one or more events is associated with at least one entity from the plurality of entities, and wherein the updated NLP model is trained using the analyzed feature data from at least a subset of the plurality of source documents and using a subset of one or more corpora of documents for the clinical trial as contextual data for the at least one entity,
wherein the updated NLP model is configured to update one or more parameters of at least one layer from the plurality of layers in response to receiving a user input representing feedback to a model output from the updated NLP model.

22. A non-transitory computer-readable storage device storing instructions that when executed by one or more processors of a computing device cause the one or more processors to perform operations comprising:
obtaining, from one or more data sources for one or more clinical studies, a plurality of source documents, wherein each source document from the plurality of source documents comprises clinical trial information of the one or more clinical studies;
identifying, for each source document in the plurality of source documents and by a natural language processing (NLP) model, a plurality of entities of the one or more clinical studies from the information related to participants of the one or more clinical studies in the source document, wherein the NLP model is trained to identify the plurality of entities by analyzing feature data of (i) the information related to the participants of the one or more clinical studies across the plurality of source documents, and (ii) one or more corpora of documents for the clinical trial related to the plurality of source documents; and
generating, based on the plurality of entities and using the analyzed feature data, an updated NLP model comprising a plurality of layers and configured to detect one or more events likely to have occurred among the plurality of entities, wherein each event from the one or more events is associated with at least one entity from the plurality of entities, and wherein the updated NLP model is trained using the analyzed feature data from at least a subset of the plurality of source documents and using a subset of one or more corpora of documents for the clinical trial as contextual data for the at least one entity,
wherein the updated NLP model is configured to update one or more parameters of at least one layer from the plurality of layers in response to receiving a user input representing feedback to a model output from the updated NLP model.
